SEQUENCE LISTING (47 sequences)

SEQ ID NO: 1 - DNA, 21 bp, Artificial sequence
  Synthetic
gcatcttattctgggtaataa

SEQ ID NO: 2 - DNA, 21 bp, Artificial sequence
  Synthetic
gcctaccagtaccagaacata

SEQ ID NO: 3 - DNA, 20 bp, Artificial sequence
  Synthetic
ggaaaccactacagccagaa

SEQ ID NO: 4 - DNA, 18 bp, Artificial sequence
  Synthetic
agcagccacctcacggta

SEQ ID NO: 5 - DNA, 24 bp, Artificial sequence
  Synthetic
acaagaatcgggtggcttccagaa

SEQ ID NO: 6 - DNA, 21 bp, Artificial sequence
  Synthetic
acctcactacacaaacagacg

SEQ ID NO: 7 - DNA, 20 bp, Artificial sequence
  Synthetic
tgttgaggacgttgcagtag

SEQ ID NO: 8 - DNA, 26 bp, Artificial sequence
  Synthetic
agcctcagaaagtcttccaagccatc

SEQ ID NO: 9 - DNA, 18 bp, Artificial sequence
  Synthetic
caacaccgcactggagag

SEQ ID NO: 10 - DNA, 19 bp, Artificial sequence
  Synthetic
gcctgcttcactttcttgg

SEQ ID NO: 11 - DNA, 23 bp, Artificial sequence
  Synthetic
aggaaaaggacatcgtcggaggc

SEQ ID NO: 12 - DNA, 23 bp, Artificial sequence
  Synthetic
tgggatgtagacaagttacgcct

SEQ ID NO: 13 - DNA, 19 bp, Artificial sequence
  Synthetic
tgctgacccttctgctgct

SEQ ID NO: 14 - DNA, 19 bp, Artificial sequence
  Synthetic
tcatggcggactgcgcctt

SEQ ID NO: 15 - DNA, 17 bp, Artificial sequence
  Synthetic
tgcatggcggtcattcc

SEQ ID NO: 16 - DNA, 17 bp, Artificial sequence
  Synthetic
catgggcaaatacgcgg

SEQ ID NO: 17 - DNA, 27 bp, Artificial sequence
  Synthetic
atgtcttccttccctcgtttcaatcag

SEQ ID NO: 18 - DNA, 20 bp, Artificial sequence
  Synthetic
tacaggacctggaaatggcc

SEQ ID NO: 19 - DNA, 18 bp, Artificial sequence
  Synthetic
tctttgaggtccactgcc

SEQ ID NO: 20 - DNA, 26 bp, Artificial sequence
  Synthetic
aggaagactcatctggaccagaaggc

SEQ ID NO: 21 - DNA, 20 bp, Artificial sequence
  Synthetic
ggagtttcccccgattcaag

SEQ ID NO: 22 - DNA, 21 bp, Artificial sequence
  Synthetic
tccatgctctcgtccacatct

SEQ ID NO: 23 - DNA, 23 bp, Artificial sequence
  Synthetic
agggcgcaagttctccggtaccc

SEQ ID NO: 24 - DNA, 21 bp, Artificial sequence
  Synthetic
cacaaaaatggcgcaagatga

SEQ ID NO: 25 - DNA, 22 bp, Artificial sequence
  Synthetic
tggtagagttgggccttcagtt

SEQ ID NO: 26 - DNA, 19 bp, Artificial sequence
  Synthetic
agaagcttcggcggtcctg

SEQ ID NO: 27 - DNA, 21 bp, Artificial sequence
  Synthetic
gttcccctgatatactcctgc

SEQ ID NO: 28 - DNA, 21 bp, Artificial sequence
  Synthetic
ggacatgaaagactgcctgag

SEQ ID NO: 29 - DNA, 25 bp, Artificial sequence
  Synthetic
aggatgtacggtctcttccaaagcc

SEQ ID NO: 30 - DNA, 21 bp, Artificial sequence
  Synthetic
gctagcagtgctacccccact

SEQ ID NO: 31 - DNA, 19 bp, Artificial sequence
  Synthetic
gtcaaatccgttggattcg

SEQ ID NO: 32 - DNA, 25 bp, Artificial sequence
  Synthetic
agccgaaaggattccaccattgtgc

SEQ ID NO: 33 - DNA, 21 bp, Artificial sequence
  Synthetic
ttctgtgacctctttgacacc

SEQ ID NO: 34 - DNA, 19 bp, Artificial sequence
  Synthetic
gcattgctctggctatcct

SEQ ID NO: 35 - DNA, 24 bp, Artificial sequence
  Synthetic
aaacatccctcctatggcagcgtc

SEQ ID NO: 36 - DNA, 22 bp, Artificial sequence
  Synthetic
gaacacgtagagatcctgacac

SEQ ID NO: 37 - DNA, 22 bp, Artificial sequence
  Synthetic
acatttggaggactgggaaata

SEQ ID NO: 38 - DNA, 24 bp, Artificial sequence
  Synthetic
tctacaaacttatcacgggcccgc

SEQ ID NO: 39 - DNA, 23 bp, Artificial sequence
  Synthetic
cttacactgggtgtactgtatgg

SEQ ID NO: 40 - DNA, 23 bp, Artificial sequence
  Synthetic
gctgtaggtctactcttgacaaa

SEQ ID NO: 41 - DNA, 24 bp, Artificial sequence
  Synthetic
ccactgacgtggatgccctaatgt

SEQ ID NO: 42 - DNA, 23 bp, Artificial sequence
  Synthetic
cccttaacgagaggaagttgtag

SEQ ID NO: 43 - DNA, 20 bp, Artificial sequence
  Synthetic
ttccttcgcggttgtagatg

SEQ ID NO: 44 - DNA, 24 bp, Artificial sequence
  Synthetic
aagatgtgaccttggcgatgacct

SEQ ID NO: 45 - DNA, 40 bp, Artificial sequence
  Synthetic
cataagcttgataagcattctcaggagcaggctgaggggc

SEQ ID NO: 46 - DNA, 40 bp, Artificial sequence
  Synthetic
tcggcgcgggtcttgtagttgccgtcgtccttgaagaaga

SEQ ID NO: 47 - DNA, 38 bp, Artificial sequence
  Synthetic
tggtgcaggaggcattgctgatgatcttgaggctgttg

(19) United States
(12) Patent Application Publication
(10) Pub. No.: US 2024/0050426 A1
(43) Pub. Date: Feb. 15, 2024

(54) TREATING VIRAL INFECTION WITH NMD INHIBITORS

(71) Applicant: The Cleveland Clinic Foundation, Cleveland, OH (US)

(72) Inventors: Michaela Gack, Cleveland, OH (US); Michiel van Gent, Cleveland, OH (US)

(21) Appl. No.: 18/546,245

(22) PCT Filed: Feb. 14, 2022
(86) PCT No.: PCT/US2022/016311

§ 371 Date: Aug. 11, 2023

Related U.S. Application Data
(60) Provisional application No. 63/149,920, filed on Feb. 16, 2021.

(51) Int. Cl. A61K 31/4745; A61K 31/498; A61K 31/522; A61K 45/06; A61P 31/22

(52) U.S. Cl. CPC A61K 31/4745; A61K 31/498; A61K 31/522; A61K 45/06; A61P 31/22

(57) ABSTRACT

Provided herein are compositions, systems, and methods treating latent viral infection with an NMD inhibitor (e.g. to reactive the latent virus to lytic virus), in combination with an anti-viral agent. In certain embodiments, the latent viral infection is caused by EBV or KSHV. In other embodiments, cancer (e.g., caused by the virus) is treated by further administering an anti-cancer agent, such as an immunomodulatory agent.

The present application claims priority to U.S. Provisional application Ser. No. 63/149,920, filed Feb. 16, 2021, which is herein incorporated by reference in its entirety.

This invention was made with government support under AI148082 awarded by the National Institutes of Health. The government has certain rights in the invention.

### SEQUENCE LISTING

The text of the computer readable sequence listing filed herewith, titled “39292-601 SEQUENCE LISTING ST25”, created Feb. 14, 2022, having a file size of 8,794 bytes, is hereby incorporated by reference in its entirety.

### FIELD OF THE INVENTION

Provided herein are compositions, systems, and methods treating latent viral infection with an NMD inhibitor (e.g. to reactive the latent virus to lytic virus), in combination with an anti-viral agent. In certain embodiments, the latent viral infection is caused by EBV or KSHV. In other embodiments, cancer (e.g., caused by the virus) is treated by further administering an anti-cancer agent, such as an immunomodulatory agent.

### BACKGROUND

Herpesviruses are large, enveloped DNA viruses that establish widespread persistent infections. The two human oncogenic gamma-herpesviruses Epstein-Barr virus (EBV) and Kaposi's sarcoma-associated herpesvirus (KSHV) are carried by a large proportion of the adult population worldwide and pose a significant risk of infection-associated morbidity and mortality, especially in immunocompromised hosts (1-3). For example, both EBV and KSHV cause a range of malignancies of lymphoid, epithelial, and endothelial origin, and KSHV remains one of the leading causes of death in HIV patients (2, 4, 5). Following primary infection, EBV and KSHV typically establish a lifelong latent infection in B cells that is characterized by the near-complete absence of viral gene expression. Occasional reactivation in a small proportion of infected cells, which involves the coordinated expression of the full repertoire of viral lytic genes, leads to the production of viral particles and transmission to new cells and hosts (6-9).

While major improvements have been made in the understanding of the viral factors involved in the latent-to-lytic switch, the molecular details of the host factors that drive viral reactivation remain poorly understood. Notably, latently-infected cells are resistant to currently available anti-herpesvirus drugs, which exclusively target the lytic phase of infection; the latent viral reservoirs therefore pose a major obstacle to eliminating persistent EBV and KSHV infection (10, 11). Furthermore, low-level viral reactivation is an important contributor to gammaherpesvirus-associated persistence and tumorigenesis (12, 13). Gaining molecular insight into the factors that modulate latency and reactivation of these viruses is thus of fundamental importance for the development of more effective therapeutic strategies to treat gammaherpesvirus-associated diseases.

NMD (nonsense-mediated RNA decay) is an evolutionarily conserved co-translational RNA degradation process that eliminates mRNA transcripts harboring premature termination codons (PTCs) or other, less common, NMD-inducing features (14, 15). Historically, NMD has been known to target faulty mRNA transcripts, which can arise through aberrant splicing or mutagenesis, for degradation to prevent the expression of nonfunctional or dominant-negative proteins that could jeopardize cellular integrity. However, more recently it has become clear that a significant portion of ‘intact’ cellular transcripts contain NMD-inducing features that allow cells to regulate their expression level and maintain homeostasis in response to environmental changes such as those encountered during development, cellular differentiation, and stress (16, 17).

An important prerequisite for PTC recognition by the NMD machinery is the splicing-dependent deposition of exon-junction complexes (EJCs), which typically contain the NMD proteins UPF2 and UPF3b, more than 50-55 nucleotides downstream of the PTC (18, 19). The presence of the EJC causes stalling of the ribosome and the translation termination complex at the PTC, which favors recruitment of the key NMD factor UPF1. Subsequent phosphorylation of UPF1 by the serine-threonine kinase SMG1 facilitates an interaction between UPF1 and UPF2/UPF3b. This triggers translational repression and targets the transcript for degradation by various (indirect) nucleolytic pathways through recruitment of additional NMD factors such as the nuclease SMG6 or the SMG5/SMG7 dimer (14). Alternatively, NMD can be initiated in an EJC-independent manner by the presence of an unusually long (>1 kb) 3′-UTR (17, 20). This process is less well-understood and is likely also induced by delayed translation termination that increases the chance of UPF1 recruitment and phosphorylation at the terminating ribosome (14).

### SUMMARY

Provided herein are compositions, systems, and methods treating latent viral infection with an NMD inhibitor (e.g. to reactive the latent virus to lytic virus), in combination with an anti-viral agent. In certain embodiments, the latent viral infection is caused by EBV or KSHV.

In other embodiments, cancer (e.g., caused by the virus) is treated by further administering an anti-cancer agent, such as an immunomodulatory agent.

In some embodiments, provide herein are methods comprising: a) administering an NMD inhibitor to a subject infected with a human herpesvirus, and b) administering a herpesvirus antiviral agent to the subject: prior to, after, or with the administering of the NMD inhibitor.

In further embodiments, the subject further has cancer caused by the human herpesvirus, and wherein the method further comprises: c) administering a cancer treatment agent to the subject: prior to, after, or with the administering of the NMD inhibitor. In certain embodiments, the cancer treatment agent comprises an immunomodulatory agent.

In particular embodiments, the human herpesvirus is EBV (Epstein-Barr virus). In other embodiments, the human herpesvirus is KSHV (Kaposi's sarcoma-associated herpesvirus). In certain embodiments, the human herpesvirus is selected from the group consisting of: HSV-1 (herpes simplex virus 1), HSV-2 (herpes simplex virus 2), VZV (varicella zoster virus), CMV (cytomegalovirus), HHV6A (human herpesvirus 6A), HHV6B (human herpesvirus 6B), and HHV7 (human herpesvirus 7).

In particular embodiments, the NMD inhibitor is selected from the group consisting of: NMDI-1, NMDI-14, and VGI. In additional embodiments, the herpesvirus antiviral comprises ganciclovir or valganciclovir. In additional embodiments, the subject is a human.

In some embodiments, provided herein are systems, kits, and compositions comprising: a) an NMD inhibitor, and b) a herpesvirus antiviral. In other embodiments, the NMD inhibitor is selected from the group consisting of: NMDI-1, NMDI-14, and VGI. In certain embodiments, the herpesvirus antiviral comprises ganciclovir. In additional embodiments, the systems, kits, and compositions further comprise: a cancer treatment agent. In some embodiments, the cancer treatment agent comprises an immunomodulatory agent.

In certain embodiments, the NMD inhibitor is NMDI-1, as shown below.

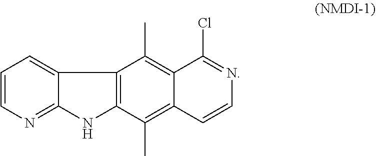 [SMILES: Cc1c2ccnc(Cl)c2c(C)c2c1[nH]c1ncccc12]

In other embodiments, the NMD inhibitor comprises VGI, as shown below.

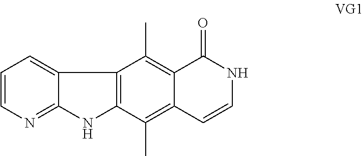 [SMILES: Cc1c2cc[nH]c(=O)c2c(C)c2c1[nH]c1ncccc12]

Gotham et al., Org Biomol Chem. 2016 Feb. 7; 14(5): 1559-1563, provides the structure of NMD-1 and VG1 (herein incorporated by reference).

In some embodiments, the NMD inhibitor comprises NMDI-14, as shown below:

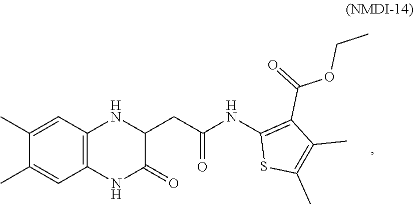 [SMILES: CCOC(=O)c1c(NC(=O)CC2Nc3cc(C)c(C)cc3NC2=O)sc(C)c1C]

available form Sigma.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1. NMD restricts spontaneous reactivation of the oncogenic herpesviruses EBV and KSHV. (A, B) Microscopy (A) and flow cytometry (B) analysis of green fluorescent protein (GFP, green) expression as a marker for EBV reactivation in AGS-EBV cells at the indicated hours post transfection (hpt) with UPF1-specific siRNAs (si.UPF1) or non-targeting control siRNAs (si.Con). Treatment with 2.5 mM sodium butyrate (NaB) for 24 h served as positive control for reactivation. DAPI, nuclear stain (blue). Scale bar, 50 (C) Abundance of lytic EBV transcripts BZLF1, BRLF1, BMRF1, and BLLF1 in AGS-EBV cells transfected with si.Con or siRNAs targeting UPF1, STAU1, or STAU2 for 96 h, determined by qRT-PCR and presented as fold expression relative to si.Con. (D, E) Microscopy (D) and flow cytometry (E) analysis of red fluorescent protein (RFP, red) expression as a marker for KSHV reactivation in HEK293T.rKSHV219 cells at the indicated hours post transfection (hpt) with si.UPF1 or si.Con. Constitutively expressed GFP (green) served as a marker for total KSHV⁺ cell number. Scale bar, 50 μm. (F) KSHV lytic Orf50, Orf74, and Orf26 levels in HEK293T.rKSHV219 cells assessed by qRT-PCR at 96 h post transfection with siRNAs specific to UPF1, STAU1 and/or STAU2, or si.Con, presented as fold abundance relative to si.Con. (G) Immunoblotting analysis of EBV Zta and EA-R as well as cellular UPF1 protein levels in AGS-EBV cells transfected with si.UPF1 or non-targeting control siRNA (si.C) for 0 to 120 h. β-actin served as loading control. See S1B Fig for quantification of effects. (H) Immunoblotting analysis of cellular UPF1 and KSHV ORF45 and K8.1 protein expressions in HEK293T.rKSHV219 cells transfected with si.UPF1 or si.Con for 96 h. p97 served as loading control. See S1C Fig for quantification of effects. (I) qRT-PCR analysis of the indicated lytic EBV genes (left panel) and UPF1 (right panel) in AKBM cells at 96 h after lentiviral transduction with an shRNA targeting UPF1 (sh.UPF1) or a non-targeting control shRNA (sh.Con), presented as fold abundance relative to sh.Con. (J) Cellular UPF1 and EBV Zta and EA-R protein abundances in AKBM cells at 120 h post transduction with two individual shRNAs targeting UPF1 (sh.UPF1 #1 and #2) or sh.Con, assessed by immunoblotting with specific antibodies. See 5D Fig for quantification of effects. (K) Quantification of relative KSHV genome copies in the supernatant of HEK293T.rKSHV219 cells following transfection with si.UPF1 (black, solid line) or si.Con (grey, dashed line) for 24-120 h, determined by qPCR with primers specific to Orf26. (L) Percentage of GFP-positive, EBV-infected HEK293T cells following 30 h incubation with the supernatant of HEK293T.rKSHV219 cells that were transfected for 96 h with si.Con or si.UPF1, determined by flow cytometry. Data are representative of at least two (B, K, L) or three (A, C, D, E, F, G, H, I, J) independent experiments. Pooled data are presented as mean±SD of at least three biological replicates; ns, p>0.05, * p<0.05, ** p<0.01, *** p<0.001; one-way ANOVA for B, C, E, F; two-sided Student's t-test for I, K, L.

FIG. 2. Gammaherpesvirus transcripts are targeted by the NMD machinery. (A) Schematic of the UPF1-affinity purification and RNAseq (RIP-seq) approach used to identify viral NMD targets. (B, C) EBV and KSHV transcript enrichment with UPF1 following UPF1 immunoprecipitation (IP) from AGS-EBV (B) or BCBL1 (C) cell extracts, respectively, determined by RNAseq analysis and presented as fold enrichment in the UPF1 IP relative to the IgG control IP (log₂ values; y-axis) versus relative abundance in the total input RNA (loge values; x-axis). Labels in plot indicate the ten viral transcripts with the greatest enrichment. (D-I) qRT-PCR analysis of the enrichment of the indicated viral transcripts with UPF1 following UPF1 IP from extracts of EBV⁺ AGS-EBV (D), AKBM (E), P3HR-1, (F) or LCL (G) cells, as well as KSHV⁺ HEK293T.rKSHV219 (H) or BCBL1 (I) cells, presented as fold abundance in the UPF1 IP relative to the IgG control IP (normalized to 18S). GAPDH, a cellular transcript that is not sensitive to NMD, served as control. Data are representative of at least two independent experiments and presented as mean±SD of three technical replicates; ns, p>0.05, ** p<0.01, *** p<0.001; one-way ANOVA.

FIG. 3. NMD controls the abundance of the polycistronic EBV transactivator transcripts and Rta protein expression. (A) Schematic of the EBV transactivator locus and the three transcripts expressed from this locus. Dashed lines mark spliced introns; CDS, coding sequence; Rp, EBV BRLF1 promoter; Zp, EBV BZLF1 promoter; pA, 3′-polyadenylation site; green bar, location of the Northern blotting probe used in F and I. (B) qRT-PCR analysis of BRLF1 transcripts in HEK293T cells transfected with 0, 20, 40, or 60 nM UPF1-targeting siRNA for 24 h, followed by transfection of a plasmid encoding the complete EBV transactivator locus (left panel, orange) or the BRLF1 coding sequence (CDS) only (right panel, blue), together with pcDNA3-nlsGFP (internal control) for 48 h. Data was normalized to GFP transcript levels to control for differences in transfection efficiency and presented as fold abundance relative to cells without si.UPF1 (−). (C) qRT-PCR analysis of BZLF1 transcripts in HEK293T cells transfected with increasing amounts of si.UPF1 for 24 has in (B), followed by transfection for 48 h with a plasmid encoding the EBV BZLF1 locus (left panel, orange) or the BZLF1 CDS (right panel, blue), together with pcDNA3-nlsGFP (internal control). Data were normalized and presented as in (B). (D) Immunoblotting analysis of Rta and cellular UPF1 protein in HEK293T cells transfected with 0, 20, 40, 60, or 80 nM si.UPF1 for 24 h, followed by transfection of a plasmid encoding the complete EBV transactivator locus for 48 h. β-actin served as loading control. See 10E Fig for quantification of effects. (E) Immunoblotting analysis of Zta and cellular UPF1 protein in HEK293T cells transfected with 0, 20, 40, or 60 nM si.UPF1 for 24 h, followed by transfection with a plasmid encoding the EBV BZLF1 locus for 48 h. β-actin served as loading control. See S6F Fig for quantification of effects. (F) Northern blotting analysis of BRLF1 and BZLF1 transcripts using the probe indicated in (A), in HEK293T cells transfected with 80 nM si.Con or si.UPF1 for 24 h, followed by transfection for 48 h with an empty control vector (−) or a plasmid encoding the complete EBV transactivator locus (+), together with pcDNA3-nlsGFP. Arrows indicate the 4, 3.3, and 1.3 kb transcripts derived from the transactivator locus. GFP served as internal control for transfection efficiency. GAPDH served as loading control. (G) qRT-PCR analysis of BRLF1 transcripts in HEK293T cells transfected with 0, 20, 40, or 60 nM si.UPF1 for 24 h, followed by transfection of a plasmid encoding the wildtype EBV transactivator locus (orange) or mutant plasmids lacking either the two BZLF1 introns (Δ3′-introns, blue) or the entire 3′-UTR (Δ3′-UTR, green), together with pcDNA3-nlsGFP, for 48 h. Data are presented as fold expression relative to the sample without si.UPF1 (−) after normalization to GFP transcript abundance. (H) Immunoblotting analysis of Rta protein in HEK293T cells transfected as in (G). See S6G Fig for quantification of effects. (I) Northern blotting analysis of transactivator transcripts in total mRNA or UPF1-associated RNA following UPF1 IP from AKBM cells, using the probe indicated in (A). Arrows mark the three transcripts derived from the transactivator locus. (J) qRT-PCR analysis of the indicated EBV transcripts in AGS-EBV cells treated with 5 ng/mL TPA without (Mock) or with 25 μM NMDI-1 for 24 h. Data are presented as fold abundance relative to the mock-treated sample. Data are representative of at least two (D, E, J) or three (B, C, F, G, H, I) independent experiments. Pooled data are presented as mean±SD of three biological replicates; ns, p>0.05, * p≤0.05, ** p<0.01, *** p≤0.001; one-way ANOVA for B, C, and G; two-sided Student's t-test for J.

FIG. 4. Small-molecule NMD inhibitor NMDI-1 is a potent inducer of EBV and KSHV reactivation. (A-C) qRT-PCR analysis of EBV BZLF1, BRLF1, BMRF1, BLLF1b, and/or BcLF1 transcripts in EBV⁺ AKBM (A), AGS-EBV (B), or LCL (C) cells treated with 25 μM NMDI-1 for 12 to 120 h, presented as fold expression relative to Mock-treated samples. (D-G) qRT-PCR analysis of Orf26, Orf50, Orf57, or Orf74 transcripts in KSHV⁺ iSLK.rKSHV219 (D), HEK293T.rKSHV219 (E), BC3 (F), and BCBL1 (G) cells treated with 25 μM NMDI-1 for 24 to 120 h, presented as fold expression relative to Mock-treated samples. Data are representative of at least two (A, C) or three (B, D, E, F, G) independent experiments and presented as mean±SD of three biological replicates; ns, p>0.05, * p≤0.05, ** p≤0.01, *** p≤0.001.

FIG. 5. Representative knockdown efficiency of UPF1, STAU1, and STAU2 as well as densitometric quantification of immunoblots presented in FIG. 1. (A) Representative qRT-PCR analysis of UPF1, STAU1, and STAU2 knockdown efficiency in AGS-EBV cells transfected with the indicated siRNAs for 96 h, displayed as fold expression relative to si.Con-transfected cells. (B-D) Densitometric quantification of the relative signal intensities in 2 or 3 independent biological replicates of the representative immunoblots presented in FIGS. 1G (B), 1H (C), and 1J (D).

FIG. 6. Silencing of NMD components induces EBV and KSHV reactivation. (A) Immunoblotting analysis of EBV proteins Zta and EA-R in AGS-EBV cells transfected for 96 h with siRNAs targeting the indicated NMD factors, or a non-targeting control siRNA (si.Con). β-actin served as loading control. Treatment with 2.5 mM sodium butyrate (NaB) for 24 h served as positive control for reactivation. (B) qRT-PCR analysis of EBV lytic genes BZLF1, BMRF1, and BcLF1 (left panel) and silencing efficiency of the respective NMD components (right panel) in AGS-EBV cells treated as in (A), presented as fold expression relative to si.Con. (C) qRT-PCR analysis of KSHV Orf50 and Orf26 transcripts (left panel) and knockdown efficiency of the respective NMD components (right panel) in HEK293T.rKSHV219 cells transfected with siRNAs targeting the indicated NMD genes for 72 h, presented as fold expression relative to si.Con. (D) qRT-PCR analysis of KSHV lytic Orf26 and Orf74 transcripts (left panel) and knockdown efficiency of the respective NMD components (right panel) in iSLK.rKSHV219 cells transfected with the indicated siRNAs for 96 h, presented as fold expression relative to si.Con. Of note, SMG1 could not be efficiently silenced in HEK293T.rKSHV219 or iSLK.rKSHV219 cells and therefore was not tested. Data are presented as mean±SD of at least three biological replicates; * p≤0.05, ** p≤0.01, *** p≤0.001.

FIG. 7. EBV and KSHV transcriptome analysis. (A) Whole genome RNAseq coverage plots (log₂ scale) of RNAseq reads mapping to the forward (orange) or reverse (blue) strands of the EBV genome. AGS-EBV cells were transfected for 96 h with UPF1-specific siRNAs (si.UPF1) or non-targeting control siRNAs (si.Con) with or without treatment with 2.5 mM NaB for the last 24 h, followed by RNA purification and whole transcriptome RNAseq analysis. Plots are aligned to a schematic representation of the EBV genome depicting annotated ORFs on the forward (orange) and reverse (blue) strands. Lower panel displays the fold induction of mapped reads of the si.UPF1 and NaB-treated samples relative to si.Con sample (log₂ scale). (B) Whole genome RNAseq coverage plots (log₂ scale) of RNAseq reads mapping to the forward (orange) or reverse (blue) strands of the KSHV genome, derived from HEK293T.rKSHV219 cell extracts treated and displayed as in (A). The RNAseq data has been deposited under NCBI BioProject accession number PRJNA677887.

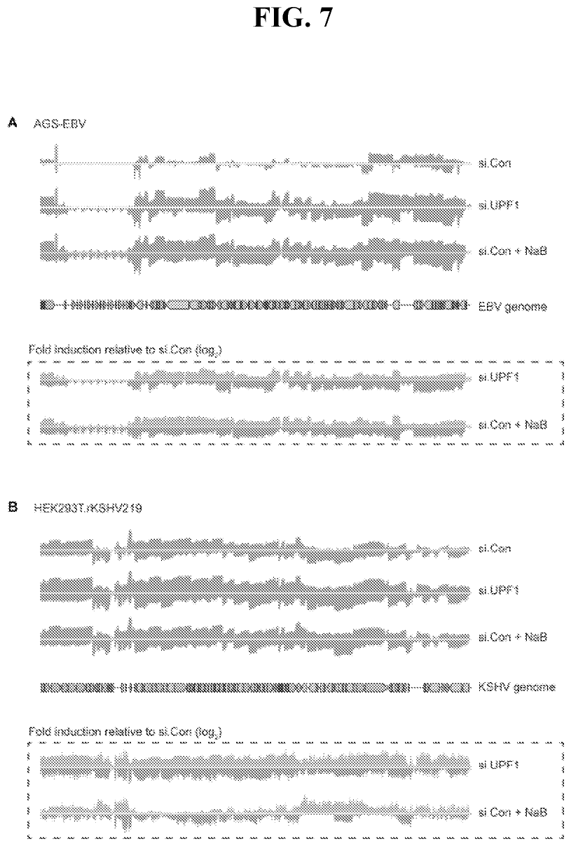

FIG. 8. RIP-seq analysis of EBV and KSHV transcripts associated with endogenous UPF1 or phospho-UPF1. (A) Overview of the ten EBV transcripts presented in FIG. 2B that were found to be significantly enriched with endogenous UPF1 in AGS-EBV cells indicating whether they are (blue circle) or are not (red circle) encoded in a viral gene cluster with a shared polyadenylation (poly(A)) site and/or derived from an intron-containing locus. (B) Enrichment of UPF1-associated EBV transcripts following UPF1 immunoprecipitation (IP) from AGS-EBV cells treated with 2.5 mM NaB for 24 h, as determined by RNAseq analysis and presented as fold enrichment in the UPF1 IP relative to the IgG control IP (log₂ values; y-axis) versus relative abundance in the total input RNA (log₂ values; x-axis). Labels in plot indicate the ten viral transcripts with the greatest enrichment. (C, D) Enrichment of UPF1-associated EBV transcripts following phospho-UPF1 IP from untreated AGS-EBV cells (C) or AGS-EBV cells treated with 2.5 mM NaB for 24 h (D), determined by RNAseq and presented as in (B). Labels in plots indicate the significantly enriched EBV transcripts. (E) KSHV transcript enrichment with UPF1 following UPF1 IP from HEK293T.rKSHV219 cell extracts, determined by RNAseq and presented as in (B). Labels in plot indicate the ten viral transcripts with the greatest enrichment. (F) Overview of the ten KSHV transcripts presented in FIG. 2C that were found to be highly enriched with endogenous UPF1 in BCBL1 cells indicating whether they are (blue circle) or are not (red circle) encoded in viral gene cluster with a shared polyadenylation (poly(A)) site and/or derived from an intron-containing locus. The RNAseq data has been deposited under NCBI BioProject accession number PRJNA677887.

FIG. 9. Validation of the UPF1 IP assay to identify NMD-targeted transcripts. (A) Representative immunoblotting analysis showing UPF1 phosphorylation following IP with UPF1 or phospho-UPF1 (p-UPF1) specific antibodies. Due to pre-treatment of the cells with the PP2a inhibitor okadaic acid, a significant proportion of UPF1 was phosphorylated in these cell extracts, as expected. (B-G) qRT-PCR analysis of known cellular NMD-sensitive transcripts GADD45B, PDRG1, and MAP3K14 as well as the NMD-insensitive controls GAPDH, RPL32, and HPRT1 in the anti-UPF1 IP (relative to the IgG control IP) in EBV⁺ AGS-EBV (B), AKBM (C), P3HR-1 (D), or LCL (E) cells as well as KSHV⁺ HEK293T.rKSHV219 (F) or BCBL1 (G) cells. Data are representative of at least two independent experiments and presented as mean±SD of three technical replicates; ns, p>0.05, * p≤0.05, *** p≤0.001; one-way ANOVA or two-sided Student's t-test.

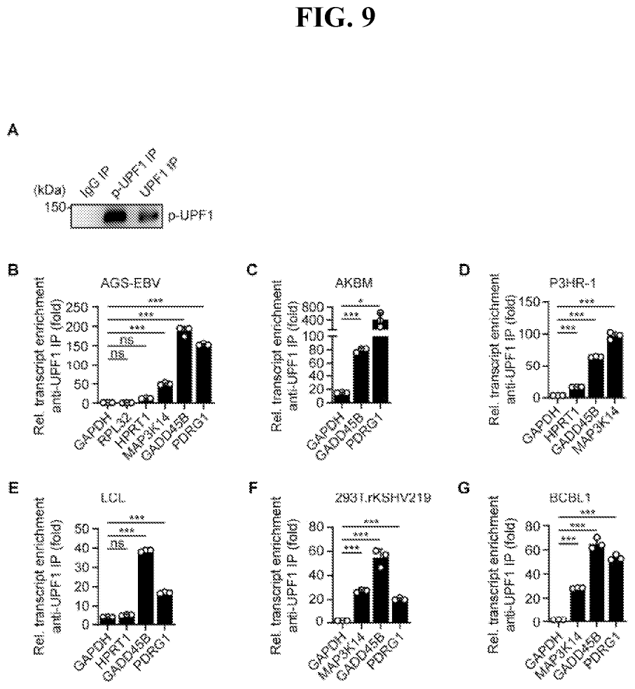

FIG. 10. NMD controls the abundance of the polycistronic EBV and KSHV transactivator transcripts. (A) qRT-PCR analysis of BRLF1 transcripts in HEK293T cells transfected with 0.5, 1.0, or 1.5 μg of a plasmid encoding the dominant-negative mutant UPF1-R843C, or an empty-vector control, together with a plasmid encoding the complete EBV transactivator locus (left panel, orange) or the BRLF1 coding sequence (CDS) only (right panel, blue) for 24 h. Data is presented as fold expression relative to the sample without UPF1-R843C (−). (B) qRT-PCR analysis of BZLF1 transcripts in HEK293T cells transfected with increasing amounts of UPF1-R843C as in (A) together with a plasmid encoding the EBV BZLF1 locus (left panel, orange) or the BZLF1 CDS (right panel, blue) for 24 h. Data is presented as fold expression relative to the control sample without UPF1-R843C (−). (C) Schematic of the KSHV transactivator locus and the seven transcripts expressed from this locus. Dashed lines mark spliced introns. CDS, coding sequence. pA, 3′ polyadenylation site. (D) qRT-PCR analysis of Orf50 transcripts in HEK293T cells transfected with 0, 20, 40, or 60 nM si.UPF1 for 24 h followed by transfection of a plasmid encoding the complete KSHV transactivator locus (left panel, orange) or the Orf50 CDS (right panel, blue) for 48 h. Data was normalized to cotransfected GFP transcript levels to control for differences in transfection efficiency, and presented as fold expression relative to si.Con-transfected cells (−). (E-G) Densitometric quantification of the relative signal intensities in 2 or 3 independent experimental replicates of the representative immunoblots presented in FIGS. 3D (E), 3E (F), and 3H (G). For the highest concentration of si.UPF1 (++++) in panel E only one replicate was included. Data in A, B, and D are representative of at least three independent experiments and presented as mean±SD of three biological replicates; ns, p>0.05, * p≤0.05, ** p≤0.01, *** p≤0.001; one-way ANOVA.

FIG. 11. Cytotoxicity assessment of NMDI-1 treatment. (A, B) Flow cytometry analysis of virus-negative Burkitt lymphoma BJAB (A) or AGS (B) cells treated with 5 to 100 μM NMDI-1 or DMSO (‘0 μM’ NMDI-1) for 48 h and stained with 7-AAD and FITC-Annexin V. Data are presented as mean percentage of live (orange, Annexin V⁻/7-AAD⁻), apoptotic (blue, Annexin V⁻/7-AAD⁻), and necrotic (red, Annexin V⁺/7-AAD⁺) cells for three biological replicates. 1 μM staurosporine treatment served as positive control for apoptosis/necrosis induction.

FIG. 12. GSEA analysis of cellular transcripts enriched with UPF1 in AGS-EBV cells. Gene Set Enrichment Analysis (GSEA) was performed by comparing cellular transcripts precipitated with endogneous UPF1 or phospho-UPF1 to those precipitated with the IgG control in untreated or NaB-treated AGS-EBV cells, as described for FIGS. 2B and 8B-8D Fig. Analysis of significantly enriched gene sets was performed using the Molecular Signatures Database and presented as the normalized enrichment scores for the fourteen gene sets that were significantly enriched in all four conditions (p-value<0.05 and false discovery rate<0.25). The RNAseq data has been deposited under NCBI BioProject accession number PRJNA677887.

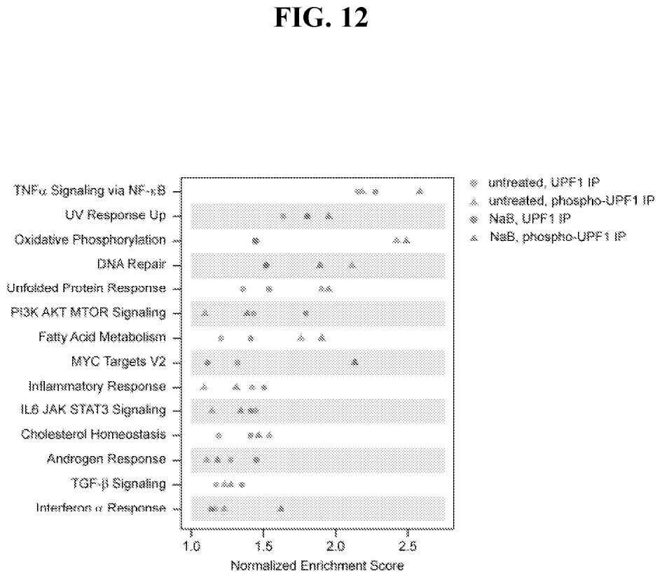

### DETAILED DESCRIPTION

Provided herein are compositions, systems, and methods treating latent viral infection with an NMD inhibitor (e.g. to reactive the latent virus to lytic virus), in combination with an anti-viral agent. In certain embodiments, the latent viral infection is caused by EBV or KSHV. In other embodiments, cancer (e.g., caused by the virus) is treated by further administering an anti-cancer agent, such as an immunomodulatory agent.

The two human oncogenic gamma-herpesviruses Epstein-Barr virus (EBV) and Kaposi's sarcoma-associated herpesvirus (KSHV) are carried by a large proportion of the adult population worldwide and are each responsible for a significant number of human cancer cases. Characteristic of herpesviruses, EBV and KSHV establish a lifelong latent infection in B cells that is characterized by the near-complete absence of viral gene expression. These latently-infected cells are resistant to the currently available anti-herpesvirus drugs and the latent viral reservoirs therefore pose a major obstacle to eliminating persistent EBV and KSHV infection. Furthermore, the far majority of virus-positive tumor cells are typically latently infected and thus resistant to anti-viral therapy. Therefore, there is a pressing interest in finding ways to therapeutically disrupt latency and induce lytic reactivation to eliminate the latent viral reservoirs, to sensitize tumor cells to antiviral drugs, and to activate cytotoxic T-lymphocyte responses that are primarily directed against lytic antigens. In word conducted during the development of embodiments herein, we identified the evolutionarily conserved cellular RNA degradation pathway nonsense-mediated decay (NMD) as a strong regulator of EBV and KSHV latency and reactivation. Treatment with a small-molecule inhibitor of NMD, NMDI-1, induced robust reactivation of EBV and KSHV in a variety of latently-infected cells types, even those that are notoriously refractory to reactivation. This indicates that NMD inhibition is a good strategy to therapeutically induce viral reactivation in the treatment of EBV and KSHV-associated morbidities and malignancies

In certain embodiments, an NMD inhibitor is administered to an infected subject to reactivate latent infection and make previously refractory cells sensitive to antivirals and cytotoxic T-cells through expression of viral antigens from lytic cells. In certain embodiments, infected subjects are administered NMD inhibitors so as to induce cell death in cancer cells by reactivating latent virus (e.g., to both lyse the cell and cytotoxic T-cell attack). In certain embodiments, this is used in combination with other immunomodulatory therapies and could provide a way to break immunosuppression. In other embodiments, the subject with has latent virus infection and is administered NMD inhibitors in combination with antiviral drugs (e.g., anti-herpesvirus drugs).

In certain embodiments, the subject is infected with Epstein-Barr virus (EBV) and/or Kaposi's sarcoma-associated herpesvirus (KSHV). Epstein-Barr Virus (EBV) is associated with several malignancies, including nasopharyngeal cancer (NPC), Burkitt's lymphoma, non-Hodgkin's lymphoma, gastric carcinoma, and NK/T cell lymphoma and is estimated to be responsible for one-to-two percent of all human cancer worldwide. Although there are treatments, many of these cancers become refractory to treatment. Additionally, headway for complete remission has been made with CART therapies for some indications but this is an extremely expensive treatment and has a long term remission rate of 30-60% depending on the treatment. There are currently no treatments available for latent infection with EBV or KSHV.

### EXAMPLES

### Example 1

### Nonsense-Mediated Decay Controls the Reactivation of the Oncogenic Herpesviruses EBV and KSHV

The oncogenic human herpesviruses Epstein-Barr virus (EBV) and Kaposi's sarcoma-associated herpesvirus (KSHV) are the causative agents of multiple malignancies. A hallmark of herpesviruses is their biphasic life cycle consisting of latent and lytic infection. In this example, we identified that cellular nonsense-mediated decay (NMD), an evolutionarily conserved RNA degradation pathway, critically regulates the latent-to-lytic switch of EBV and KSHV infection. The NMD machinery suppresses EBV and KSHV Rta transactivator expression and promotes maintenance of viral latency by targeting the viral polycistronic transactivator transcripts for degradation through the recognition of features in their 3′-UTRs. Treatment with a small-molecule NMD inhibitor potently induced reactivation in a variety of EBV- and KSHV-infected cell types. These results identify NMD as an important host process that controls oncogenic herpesvirus reactivation, which may be targeted for the therapeutic induction of lytic reactivation and the eradication of tumor cells and viral infection.

### Methods

### Cell Culture

HEK293T cells (ATCC) were maintained in Dulbecco's Modified Eagle's Medium (DMEM, Gibco) supplemented with 10% (v/v) heat-inactivated fetal bovine serum (FBS), 2 mM GlutaMAX (Gibco), and 1% (v/v) penicillin-streptomycin (Pen-Strep, Gibco) under standard tissue culture conditions. HEK293T.rKSHV219 and iSLK.rKSHV219 cells (kindly provided by D. Ganem, University of California) were maintained in DMEM supplemented with 10% (v/v) FBS, 2 mM GlutaMAX, 1% (v/v) Pen-Strep, and 2 μg/mL puromycin (Sigma). AGS (ATCC) and AGS-EBV cells (kindly provided by N. Raab-Traub, University of North Carolina, Chapel Hill (32)) were cultured in F-12 nutrient mixture (Gibco) supplemented with 10% FBS, 2 mM GlutaMAX, 1% (v/v) Pen-Strep, and 500 μg/mL G418 (Sigma). BJAB, P3HR-1 (kindly provided by B. Gewurz, Harvard Medical School), and AKBM cells (kindly provided by M. Ressing, Leiden University Medical Center, The Netherlands (35)) were cultured in Roswell Park Memorial Institute medium (RPMI, Gibco) supplemented with 10% (v/v) FBS, 2 mM GlutaMAX, 1% (v/v) Pen-Strep, and 0.3 mg/mL hygromycin B. The PEL cells BC3 and BCBL1 were cultured in RPMI supplemented with 20% (v/v) FBS, 2 mM GlutaMAX and 1% (v/v) Pen-Strep. LCL cells were prepared by incubating human healthy donor-derived CD19⁺ B cells (iXCells) with EBV⁺ supernatant from sodium butyrate-treated AGS-EBV cells. The LCL phenotype of the outgrowing cells was confirmed by flow cytometry analysis of CD19 expression and immunoblotting analysis of EBV EBNA1 expression. LCLs were maintained in RPMI supplemented with 10% (v/v) FBS, 2 mM GlutaMAX, and 1% (v/v) Pen-Strep.

### Plasmids and Transfections

pcDNA3-nlsGFP was kindly provided by M. Ressing (Leiden University Medical Center, The Netherlands). UPF1 was subcloned with an N-terminal HA tag from pCMV6-UPF1-MYC-DDK (Origene, NM 001297549) into a dual promoter lentiviral vector (BIC-PGK-Zeo-T2a-mAmetrine (63); kindly provided by R. J. Lebbink, University Medical Center Utrecht, The Netherlands) under control of the human EF1A promoter. To generate the dominant-negative UPF1-R843C mutant, a gBlock (IDT) encoding the required mutation was used to replace the corresponding region of UPF1 using the SbfI and Pm1I restriction sites and the Gibson Assembly method.

All EBV and KSHV transactivator-encoding plasmids were generated using Gibson assembly in a pCMV6 vector backbone from which the CMV-IE promoter was removed. All fragments used for cloning, as indicated below, were either generated by PCR or ordered as gBlocks (IDT) using sequences from the EBV Akata genome (Genbank accession number KC207813.1) or KSHV JSC-1 BAC16 genome (Genbank accession number GQ994935) as templates. For the plasmid containing the entire EBV transactivator locus, a fragment corresponding to nucleotides (nt) 94,567 to 89,497 of the EBV Akata genome, encompassing the entire region from the start of the Rp promoter until the polyadenylation site, was introduced into the pCMV6 backbone. The mutant ‘Δ3’-introns' plasmid was generated by deleting the two introns corresponding to nt 89,797 to 89,713 and 90,053 to 89,903 of the EBV genome. To generate the Δ3′-UTR plasmid, the entire sequence between the BRLF1 stop codon and the polyadenylation site was deleted. To obtain the BRLF1 CDS-only plasmid, the sequence downstream of the Rp promoter in the full-length plasmid was replaced with the BRLF1 coding sequence only, corresponding to nt 92,582 to 90,765 of the EBV Akata genome. To generate the plasmid encoding the BZLF1 locus, a fragment corresponding to nt 91,145 to 89,497 of the EBV genome, encompassing the region from the start of the Zp promoter until the polyadenylation site, was introduced into the pCMV6 backbone. For the BZLF1 CDS-only plasmid, the three BZLF1 exons corresponding to nt 90,554 to 90,054, nt 89,902 to 89,798, and nt 89,712 to 89,581 of the EBV genome were joined together downstream of the Zp promoter. To generate the plasmid containing the entire KSHV transactivator locus, a fragment corresponding to nt 68,333 to 76,595 of the BAC16 genome, encompassing the entire region from the start of the Orf50 promoter until the polyadenylation site, was introduced into the pCMV6 backbone lacking the CMV-IE promoter. To obtain the Orf50 CDS-only control plasmid, the two Rta-encoding Orf50 exons corresponding to nt 71,412 to 71,429 and 72,388 to 74,445 in the KSHV genome were joined together downstream of the Orf50 promoter sequence. The sequence of all constructs was verified by Sanger sequencing.

To assess transactivator transcript and protein expression levels, HEK293T cells were transfected with increasing amounts (0 nM, 20 nM, 40 nM, 60 nM, or 80 nM) of UPF1-targeting siRNAs, supplemented with non-targeting control siRNAs to 80 nM, using Lipofectamine RNAiMAX transfection reagent in 12-well plates following the manufacturer's instructions. After 24 hours, plasmid transfections were performed using 1.6 μg plasmid DNA (100 ng plasmid of interest, 100 ng pcDNA3-nlsGFP, and 1400 ng empty pCMV6 control vector) per well using Lipofectamine2000 (Life technologies) or polyethylenimine (PEI; Polysciences) according to the manufacturer's instructions. Forty-eight hours after DNA transfection, the cells were harvested for qRT-PCR or immunoblotting analysis.

siRNA-Mediated Silencing

Transient knockdown of endogenous genes was achieved by transfection of the indicated target cells with 80 nM of gene-specific siRNAs using Lipofectamine RNAiMAX transfection reagent (Life Technologies) in 12-well plates according to the manufacturer's instructions. siRNAs targeting the following genes were purchased as siGENOME SMARTpools from Dharmacon: UPF1 (M-011763-01), STAU1 (M-011894-01), STAU2 (M-006873-00), UPF2 (M-012993-01), UPF3a (M-012872-00), UPF3b (M-012871-00), SMG1 (M-005033-01), SMG5 (M-014023-00), SMG6 (M-017845-01), SMG7 (M-021305-01), and a non-targeting control (D-001206-14). After 24 to 120 hours, the cells were harvested for further analysis as indicated. Knockdown efficiency was assessed in each experiment by measuring transcript or protein abundance by qRT-PCR or immunoblotting.

### Lentiviral Transduction of AKBM Cells

Standard lentivirus production methods were used to generate in HEK293T cells third generation lentiviral particles for two shRNAs targeting UPF1 (Sigma TRC human genome wide shRNA library; pLKO.1 backbone; #1 5′-GCATCTTATTCTGGGTAATAA-3′ (SEQ ID NO:1) and #2 5′-GCCTACCAGTACCAGAACATA-3′, SEQ ID NO:2) as well as a non-targeting control shRNA. Transduction of AKBM cells was performed by adding 1 mL of lentivirus preparation to 0.5 million AKBM cells in a 12-well plate. Forty-eight hours later, cells were selected using 0.4 μg/mL puromycin, and samples were harvested at the indicated times for analysis of viral gene expression by qRT-PCR or protein expression by immunoblotting analysis.

Quantitative Real-Time PCR (qRT-PCR)

Total RNA was extracted from cells using the E.Z.N.A. HP Total RNA Isolation Kit (OMEGA Bio-Tek) according to the manufacturer's instructions. Reverse transcription and qRT—PCR were performed using equal amounts (50-500 ng) of the purified RNA and the SuperScript III Platinum One-Step qRT-PCR kit with ROX (Invitrogen) on a 7500 Fast Real-Time PCR Machine (Applied Biosystems). Premixed master mixes of TaqMan primers and probes for the detection of human transcripts were purchased from Applied Biosystems (18S) or IDT (GAPDH, UPF1, UPF2, UPF3a, UPF3b, SMG1, SMG5, SMG6, SMG7, STAU1, STAU2, GADD45B, PDRG1, RPL32, HPRT1, MAP3K14). To detect viral genes, the following custom PrimeTime primer/probe mixes were ordered from IDT: EBV BZLF1 (forward primer 5′-GGAAACCACTACAGCCAGAA-3′ (SEQ ID NO:3), reverse primer 5′-AGCAGCCACCTCACGGTA-3′(SEQ ID NO:4), probe 5′-ACAAGAATCGGGTGGCTTCCAGAA-3′(SEQ ID NO:5)), BRLF1 (forward primer 5′-ACCTCACTACACAAACAGACG-3′(SEQ ID NO:6), reverse primer 5′-TGTTGAGGACGTTGCAGTAG-3′(SEQ ID NO:7), probe 5′-AGCCTCAGAAAGTCTTCCAAGCCATC-3′(SEQ ID NO:8)), BMRF1 (forward primer 5′-CAACACCGCACTGGAGAG-3′(SEQ ID NO:9), reverse primer 5′-GCCTGCTTCACTTTCTTGG-3′(SEQ ID NO:10), probe 5′-aggaaaaggacatcgteggaggc-3′(SEQ ID NO:11)), BLLF1 (forward primer 5′-TGGGATGTAGACAAGTTACGCCT-3′(SEQ ID NO:12), reverse primer 5′-TGCTGACCCTTCTGCTGCT-3′(SEQ ID NO:13), probe 5′-tcatggcggactgcgcctt-3′(SEQ ID NO:14)), BcLF1 (forward primer 5′-TGCATGGCGGTCATTCC-3′(SEQ ID NO:15), reverse primer 5′-CATGGGCAAATACGCGG-3′(SEQ ID NO:16), probe 5′-atgtatccttccctcgtttcaatcag-3′(SEQ ID NO:17)), BKRF1 (forward primer 5′-TACAGGACCTGGAAATGGCC-3′(SEQ ID NO:18), reverse primer 5′-TCTTTGAGGTCCACTGCC-3′(SEQ ID NO:19), probe 5′-aggaagactcatctggaccagaaggc-3′(SEQ ID NO:20)), BNRF1 (forward primer 5′-GGAGTTTCCCCCGATTCAAG-3′(SEQ ID NO:21), reverse primer 5′-TCCATGCTCTCGTCCACATCT-3′(SEQ ID NO:22), probe 5′-AGGGCGCAAGTTCTCCGGTACCC-3′(SEQ ID NO:23)); and KSHV Orf50 (forward primer 5′-CACAAAAATGGCGCAAGATGA-3′(SEQ ID NO:24), reverse primer 5′-TGGTAGAGTTGGGCCTTCAGTT-3′(SEQ ID NO:25), probe 5′-AGAAGCTTCGGCGGTCCTG-3′(SEQ ID NO:26)), Orf74 (forward primer 5′-gttcccctgatatactcctgc-3′(SEQ ID NO:27), reverse primer 5′-GGACATGAAAGACTGCCTGAG-3′(SEQ ID NO:28), probe 5′-aggatgtacggtctcttccaaagcc-3′(SEQ ID NO:29)), Orf26 (forward primer 5′-GCTAGCAGTGCTACCCCCACT-3′(SEQ ID NO:30), reverse primer 5′-gtcaaatccgttggattcg-3′(SEQ ID NO:31), probe 5′-AGCCGAAAGGATTCCACCATTGTGC-3′(SEQ ID NO:32)), Orf6 (forward primer 5′-TTCTGTGACCTCTTTGACACC-3′(SEQ ID NO:33), reverse primer 5′-GCATTGCTCTGGCTATCCT-3′(SEQ ID NO:34), probe 5′-AAACATCCCTCCTATGGCAGCGTC-3′(SEQ ID NO:35)), Orf? (forward primer 5′-GAACACGTAGAGATCCTGACAC-3′(SEQ ID NO:36), reverse primer 5′-ACATTTGGAGGACTGGGAAATA-3′(SEQ ID NO:37), probe 5′-TCTACAAACTTATCACGGGCCCGC-3′(SEQ ID NO:38)), Orf71 (forward primer 5′-CTTACACTGGGTGTACTGTATGG-3′(SEQ ID NO:39), reverse primer 5′-GCTGTAGGTCTACTCTTGACAAA-3′(SEQ ID NO:40), probe 5′-CCACTGACGTGGATGCCCTAATGT-3′(SEQ ID NO:41)), Orf73 (forward primer 5′-CCCTTAACGAGAGGAAGTTGTAG-3′(SEQ ID NO:42), reverse primer 5′-TTCCTTCGCGGTTGTAGATG-3′(SEQ ID NO:43), probe 5′-AAGATGTGACCTTGGCGATGACCT-3′(SEQ ID NO:44)). Abundance of target genes was calculated by normalizing for cellular 18S or GAPDH using the Comparative CT (ΔΔCt) method. Data was displayed as mean fold expression (+/−SD) compared to control samples, which were set to 1.

For quantification of KSHV viral genome copies, cell supernatant was treated with DNase to eliminate free, non capsid-associated DNA, followed by purification of viral DNA using the QIAamp MinElute Virus Vacuum Kit (Qiagen). The purified DNA was subsequently used as input for the qPCR reactions using Orf26-specific primers.

### Immunoblotting Analysis

Cell lysates were prepared in NP-40 buffer (150 mM NaCl, 1% (v/v) NP-40, 50 mM HEPES pH 7.4, and protease inhibitor cocktail (Sigma)) or RIPA buffer (25 mM Tris HCl pH 7.6, 150 mM NaCl, 1% (v/v) NP-40, 1% (wt/v) sodium deoxycholate, 0.1% (wt/v) SDS, and protease inhibitor cocktail (Sigma)). Cell debris was pelleted by centrifugation at >13,000×g for 20 min at 4° C. and proteins were denatured by incubation at 95° C. for 2 min in 1×Laemmli Sample Buffer (BioRad). Samples were resolved by Bis-Tris-PAGE using the Mini-Protean Tetra Cell system (BioRad) and 1×MOPS-SDS running buffer (Alfa Aesar), followed by transfer onto poly-vinylidene difluoride (PVDF) membranes (Bio-Rad) using a Novex semidry transfer cell (Invitrogen) or Mini Trans-Blot Cell wet transfer system (BioRad). After blocking with 5% (wt/v) nonfat dry milk in PBS-Tween 20 for 1 h at room temperature (RT), membranes were probed with primary antibodies for either 1 h at RT or at 4° C. overnight. The primary antibodies used were: anti-Zta (1:500, sc-53904, Santa-Cruz), anti-Rta (1:250, 8C12, provided by R. Feederle, Helmholtz Zentrum München, Germany), and anti-EA-R (1:250, sc-56979, Santa Cruz) to detect EBV proteins; anti-ORF45 (1:200, sc-53883, Santa Cruz) and anti-K8.1 (1:200, sc-65446, Santa Cruz) to detect KSHV proteins; and anti-UPF1 (1:2000, D15G6, Cell Signaling Technology), anti-phosphoUPF1 (Ser1127, 1:1000, 07-1016, Millipore Sigma), anti-p97 (1:2000, 612183, BD Biosciences), and anti-β-actin (1:10,000, AC-15, Sigma) to detect cellular proteins. Next, membranes were incubated with goat anti-mouse or goat anti-rabbit horseradish peroxidase (HRP)-conjugated secondary antibodies (1:2000, #7076S and #7074S, Cell Signaling Technology) for 1 h at RT. Protein bands were visualized using the enhanced SuperSignal West Pico or Femto chemiluminescence reagent (Thermo Fisher) and were detected using a LAS Imagequant 4000 luminescent imaging system (GE Lifesciences). Densitometric quantification was performed using ImageQuant TL software (GE Lifesciences).

### Northern Blotting Analysis

Total mRNA was purified from cell pellets using the Dynabeads mRNA DIRECT Purification kit (ThermoFisher Scientific) according to the manufacturer's instructions. RNA was denatured at 70° C. for 5 min in 1×RNA loading dye (Thermo Fisher Scientific) and resolved on a 1.5% agarose gel using a Mini-Protean Tetra Cell system (BioRad) in 1×TBE buffer (Invitrogen). After electrophoresis, the RNA was transferred onto a BrightStar Plus positively charged Nylon membrane (Invitrogen) using a Criterion cell (BioRad) in 0.5×TBE buffer and UV-crosslinked to the membrane at 300 mJ/cm². The membrane was hybridized with biotinylated probes purchased from IDT, recognizing the EBV transactivator transcripts (5′-CATAAGCTTGATAAGCATTCTCAGGAGCAGGCTGAGGGGC-3′ (SEQ ID NO:45)), GFP (5′-TCGGCGCGGGTCTTGTAGTTGCCGTCGTCCTTGAAGAAGA-3′ (SEQ ID NO:46)), or GAPDH (5′-tggtgcaggaggcattgctgatgatcttgaggctgttg-3′ (SEQ ID NO: 47), in ULTRAhyb-Oligo Hybridization Buffer (Invitrogen) at 42° C. overnight, followed by incubation with a streptavidin-alkaline phosphatase conjugate for detection (Invitrogen). Imaging was performed using CDP-Star luminescent substrate (Life Technologies) and bands were detected using a LAS Imagequant 4000 luminescent imaging system (GE Lifesciences).

### Large-Scale UPF1 Immunoprecipitation

Approximately 25×10⁶ cells per sample were seeded into 15-cm dishes and either left untreated or treated with 2.5 mM sodium butyrate (NaB) for 24 hours as indicated. Next, the cells were treated with 100 nM PP2a inhibitor okadaic acid (Cell Signaling Technology) for 3 h, after which lysates were prepared in NP40 lysis buffer (50 mM HEPES, pH 7.4, 150 mM KCl, 1 mM Na₃VO₄, 0.5% (v/v) NP-40, and 0.5 mM Dithiothreitol, supplemented with protease inhibitor (Sigma)) and incubated rotating at 4° C. for 30 min. The lysates were cleared by centrifugation at 13,000×g for 20 min at 4° C. Dynabeads Protein A (Invitrogen) were precoupled with anti-UPF1 (D15G6, Cell Signaling Technology), anti-phosphoUPF1 (Ser1127, Millipore Sigma), or normal rabbit-IgG control (Millipore Sigma) antibodies overnight and mixed with the cleared lysates followed by incubation at 4° C. for 4 h with constant agitation. Precipitates were washed three times with NP-40 lysis buffer and twice with high-salt NP40 lysis buffer (containing 300 mM KCl), followed by protein digestion with proteinase K (NEB) for 30 min at 55° C. and purification of precipitated RNA using phenol/chloroform/isoamylalcohol (Sigma) according to the manufacturer's instructions.

### RNAseq Analysis

RNA samples were submitted to The University of Chicago Genomics Facility for library preparation and sequencing on a HiSeq4000 instrument using 50-base pair single-end reading (Illumina). Two (for EBV) or one (for KSHV) sets of at least three pooled independent experiments were separately processed and sequenced. The sequencing data were uploaded to the Galaxy web platform and the public server at usegalaxy.org was used for analysis (64). Raw sequence reads were quality trimmed using TRIM Galore! (Galaxy Version 0.6.3) and aligned to the human genome (Gencode GRCh38.p12 v31) as well as the EBV Akata genome (GenBank accession number KC207813.1) for AGS-EBV and AKBM samples, the KSHV JSC-1 BAC16 genome (Genbank accession number GQ994935.1) for HEK293T.rKSHV219 samples, or the KSHV GK18 genome (Genbank accession number NC 009333.1) for BCBL1 samples, using HISAT2 (Galaxy Version 2.1.0) (65). FeatureCounts (Galaxy Version 1.6.4, (66)) was used to calculate transcript abundance and significantly enriched genes were determined using DESeq2 (Galaxy Version 2.11.40.6, (67)). Coverage at individual genome positions for the whole transcriptome analysis was calculated using SAMtools mpileup (68). Graphs were generated using GraphPad Prism software. For the Gene-Set Enrichment Analysis (GSEA), transcripts enriched in the phospho-UPF1 and UPF1 IP samples from untreated and NaB-treated AGS-EBV cells were analyzed using GSEA version 4.1.0 against the Molecular Signatures Database (MSigDB; version 7.2) and compared to those of the IgG control IP samples. Significant gene sets were identified for each of the four comparisons as having a p-value<0.05 and false discovery rate<0.25.

### Fluorescence Microscopy and Flow Cytometry

AGS-EBV and HEK293T.rKSHV219 cells were seeded into 12-well plates and transfected with UPF1-specific or non-targeting control siRNAs for 24 to 120 h as indicated. As a positive control for reactivation, cells were treated with 2.5 mM NaB for 24 h. For microscopy, cells were incubated with Hoechst nucleic acid stain (1:2000, Invitrogen) in PBS for 5 min at RT, after which DAPI, as well as GFP or RFP were imaged using a fluorescence microscope (Omano). For flow cytometry analysis of GFP and RFP expression, cells were fixed in 4% paraformaldehyde in PBS and analyzed on an LSRFortessa (BD Biosciences). For the determination of the percentage of apoptotic and necrotic cells, cells were incubated with 7-AAD Viability Staining Solutions (BioLegend) and FITC-Annexin V (BioLegend) for 15 min at RT following the manufacturer's instructions, followed by analysis on a BD FACSAriaII (BD Biosciences). Data were analyzed using the Flowjo software (BD Biosciences).

### NMDI-1 Treatment

A stock concentration of the NMD inhibitor NMDI-1 (47) was maintained at 10 mM in DMSO. To determine the effect of NMDI-1 treatment on EBV and KSHV reactivation, the indicated cells were seeded into 12-well plates. The next day, the cells were treated with 25 μM of NMDI-1, or the equivalent amount of DMSO as mock treatment. The cells were harvested at the indicated times for analysis of EBV and KSHV lytic gene expression by qRT-PCR.

### Statistical Analysis

All pooled data were presented as means±SD of at least three biological replicates and analyzed using GraphPad Prism software. One-way ANOVA with Dunnett's multiple comparisons or a two-tailed Student's t test was used to test for statistical significance as indicated in the figure legends.

### Results

### NMD Restricts Spontaneous Reactivation of the Oncogenic Herpesviruses EBV and KSHV.

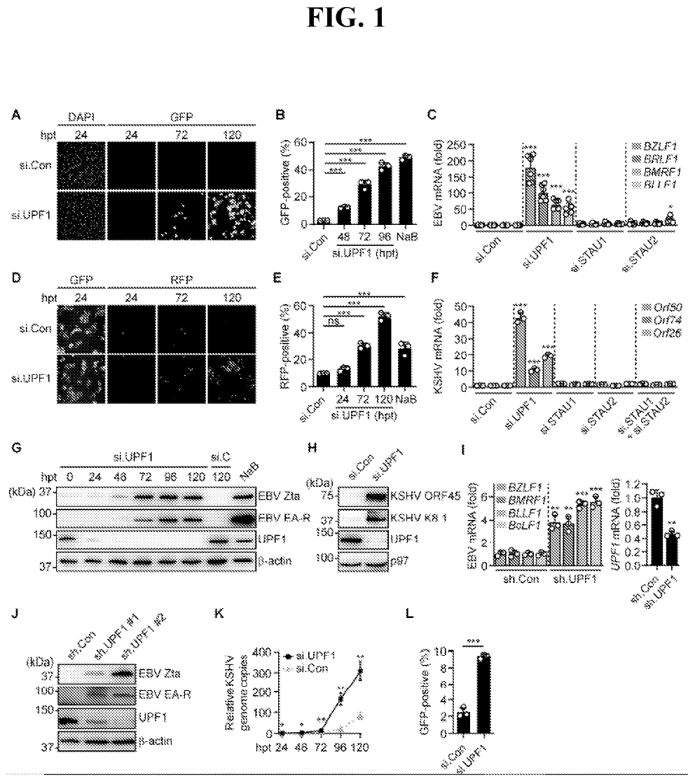

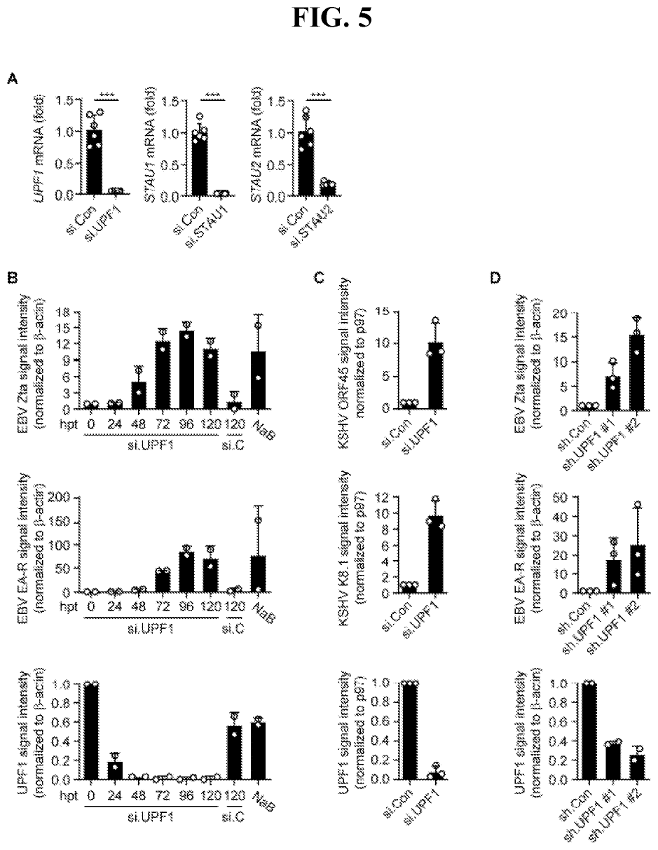

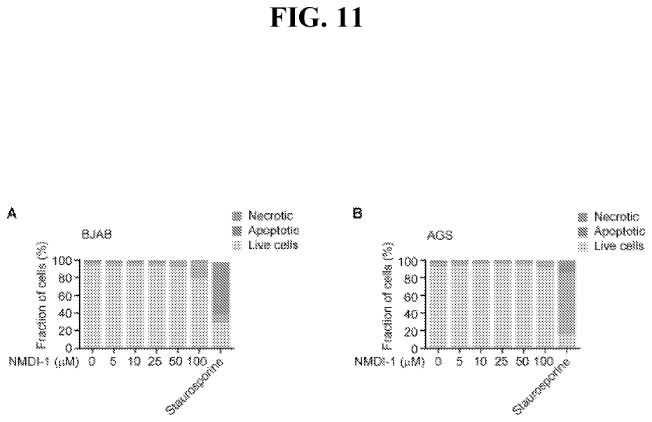

The oncogenic human herpesviruses EBV and KSHV encode many spliced, polycistronic transcripts that typically display the primary features of canonical NMD targets, such as a stop codon upstream of an EJC and/or a long 3′-UTR downstream of the proximal ORF (28-31). We therefore hypothesized that NMD regulates maintenance of viral latency and/or lytic reactivation by controlling the expression of certain gammaherpesvirus transcripts. To test this, we started by determining the effect of siRNA-mediated depletion of the critical NMD component UPF1 on spontaneous EBV reactivation in the human gastric carcinoma cell line AGS-EBV, which harbors recombinant EBV Akata-BX1 that encodes GFP under control of the lytic BXLF1 promoter (32). Fluorescence microscopy and flow cytometry analyses of AGS-EBV cells showed that UPF1 silencing markedly increased the proportion of cells expressing GFP, a measure of EBV reactivation, to a similar extent as treatment with sodium butyrate (NaB), a known potent inducer of EBV reactivation that served as a positive control (FIGS. 1A and 1B). Furthermore, evaluation of EBV lytic gene expression by qRT-PCR showed robust upregulation of EBV lytic genes BZLF1, BRLF1, BMRF1, and BLLF1 following UPF1 depletion (FIG. 1C and S1A Fig). Notably, silencing of Staufen 1 or 2 (STAU1 or STAU2), critical components of the Staufen-mediated RNA degradation pathway that is related to NMD and also critically relies on UPF1 (33), did not substantially induce lytic gene expression (FIGS. 1C and 5A Fig). In parallel, we tested the effect of NMD inhibition on KSHV reactivation in HEK293T.rKSHV219 cells, which are latently infected with recombinant KSHV.219 that constitutively expresses GFP and encodes RFP under control of the viral lytic PAN promoter (34). UPF1 silencing in these cells resulted in a robust increase in the proportion of cells expressing RFP, a marker for KSHV reactivation (FIGS. 1D and 1E), and efficiently induced expression of the KSHV lytic genes Orf50, Orf74, and Orf26 (FIG. 1F). Silencing of STAU1 or STAU2 alone, or in combination, did not induce KSHV lytic gene expression (FIG. 1F). We also observed that UPF1 depletion resulted in strongly enhanced expression of the EBV lytic proteins Zta and EA-R in AGS-EBV cells as well as KSHV lytic proteins ORF45 and K8.1 in HEK293T.rKSHV219 cells (FIG. 1G and 1H, 5B and 5C Fig). Next, we asked whether robust reactivation of EBV upon UPF1 silencing was also observed in other relevant cell types, such as the human Akata EBV⁺ Burkitt lymphoma AKBM cells (35). We found that shRNA-mediated depletion of endogenous UPF1 resulted in significant upregulation of EBV lytic genes BZLF1, BMRF1, BLLF1, and BcLF1 as well as an increased abundance of the lytic proteins Zta and EA-R (FIGS. 11 and 1J, FIG. 4D).

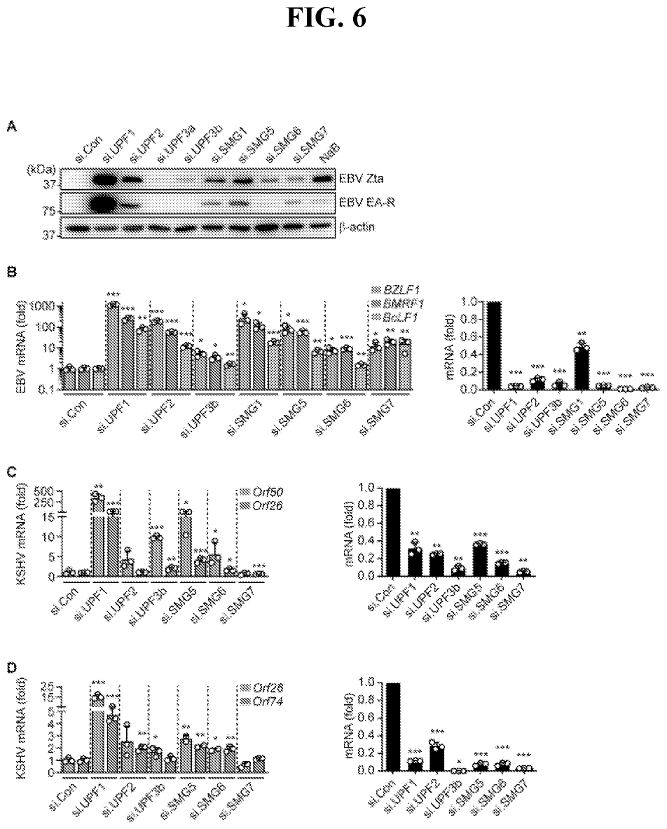

Importantly, depletion of the other NMD components UPF2, UPF3b, SMG1, SMG5, SMG6, or SMG7, also induced EBV lytic gene and protein expression (although with different potencies) in AGS-EBV cells, as well as KSHV lytic gene expression in HEK293T.rKSHV219 and iSLK.rKSHV219 cells, demonstrating that viral reactivation is induced by general interference with NMD activity rather than UPF1 silencing specifically (FIG. 5A-6D).

EBV and KSHV reactivation is a highly regulated process that consists of sequential expression of viral immediate-early transactivators, early genes, and late genes that ultimately results in the production of infectious viral particles (6, 7, 36). However, under some conditions, reactivation is abortive, characterized by limited viral lytic gene expression and the absence of viral particle production (37, 38). To determine whether NMD inhibition induces bona-fide, productive EBV and KSHV reactivation, we performed whole viral transcriptome analysis by RNAseq and observed that UPF1 depletion enhanced transcriptional activity along the entire viral genome for both EBV and KSHV, similar to NaB treatment (7A and 7B Fig). We also observed that UPF1 silencing in HEK293T.rKSHV219 cells resulted in enhanced production of KSHV particles released into the supernatant as compared to control cells (FIG. 1K) and, correspondingly, a greater infectivity when this supernatant was used to infect naïve HEK293T cells (FIG. 1L).

Taken together, these results demonstrate that inhibition of NMD robustly induces productive EBV and KSHV reactivation in various latently-infected cell types, resulting in the enhanced production of infectious virus particles.

### Gammaherpesvirus Transcripts are Targeted by the NMD Machinery.

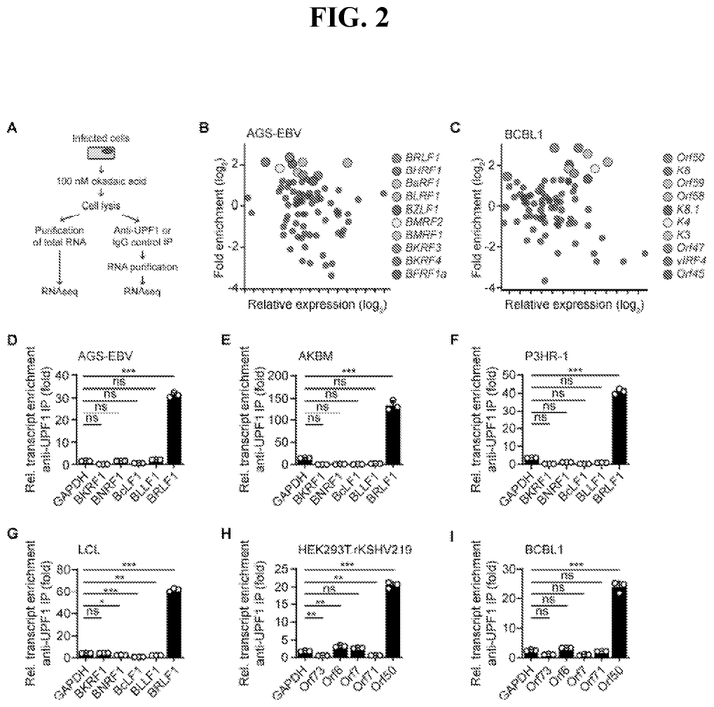

Although many cellular NMD-targeted transcripts have been identified, it remains largely obscure which viral transcripts are degraded by NMD, in particular those expressed by DNA viruses. Thus, we next sought to identify the EBV and KSHV transcripts that are targeted by NMD using an UPF1-RNA immunoprecipitation-RNAseq (RIP-seq) approach that has been commonly used to identify cellular NMD targets (39, 40). Latently infected AGS-EBV cells were pretreated with the protein phosphatase 2a inhibitor okadaic acid to inhibit dephosphorylation of UPF1 and promote the interaction between (phospho-)UPF1 and NMD-targeted transcripts. Cell extracts were prepared and immunoprecipitations were performed using anti-UPF1, or IgG as a negative control, followed by the purification of UPF1 (or IgG) associated RNAs and RNAseq analysis (FIG. 2A). In untreated AGS-EBV cells, we found ten viral transcripts to be significantly enriched with UPF1 (relative to the IgG control IP) of which the EBV transactivator-encoding BRLF1 transcript was most highly enriched with UPF1 (FIG. 2B). BZLF1 which is encoded by the polycistronic BRLF1 transactivator transcript of EBV as well as by a separate monocistronic transcript, was also among the top five enriched genes. Of note, all except one of the significantly enriched viral transcripts were part of EBV gene clusters that share a common polyadenylation site and give rise to polycistronic transcripts, including the BaRF1-BMRF1-BMRF2 and BKRF3-4 clusters (8A FIG.). Importantly, the enrichment of viral transcripts with UPF1 did not correlate with their overall abundance in the infected cell (FIG. 2B, x-axis). In parallel, this RIP-seq assay was also performed with extracts from AGS-EBV cells treated with NaB for 24 h to induce lytic reactivation and thereby increase the abundance of lytic transcripts. Under these conditions, we found BRLF1 to be the third most highly enriched viral transcript (8B). Furthermore, we performed the same analysis following precipitation of phosphorylated UPF1 (p-UPF1) using a phospho-specific antibody from untreated and NaB-treated AGS-EBV cells. While this resulted in fewer significantly enriched viral transcripts overall, BRLF1 was still the top viral transcript enriched with p-UPF1 in untreated cells, and the fourth highest enriched transcript in NaB-treated cells (8C and 8D).

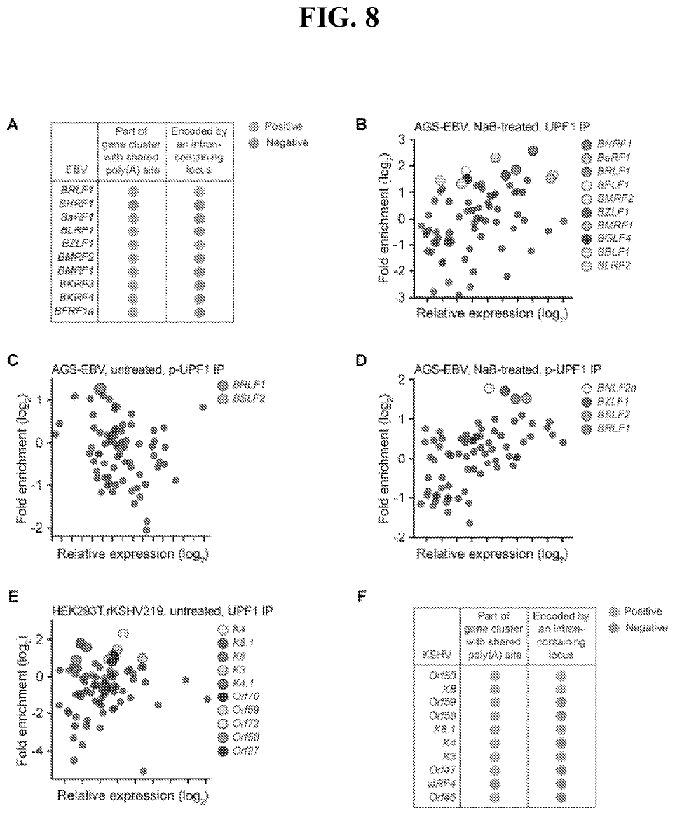

Similarly, we assessed the enrichment of KSHV transcripts with UPF1 in KSHV⁺ primary effusion lymphoma (PEL) BCBL1 and HEK293T.rKSHV219 cells. Analogous to our observations for EBV, we found that the polycistronic KSHV transactivator-encoding Orf50 transcript was the viral transcript most highly enriched with UPF1 in BCBL1 cells, and among the top ten enriched viral transcripts in HEK293T.rKSHV219 cells (FIGS. 2C and 8E). Furthermore, K8 and K8.1, which are encoded by the polycistronic Orf50 transcript, and also smaller transcripts similar to BZLF1 for EBV, were among the top enriched genes with UPF1 in both KSHV⁺ cell lines tested. Similar to our data for EBV transcripts, all except one of the highly enriched KSHV transcripts were derived from gene clusters with a shared polyadenylation site (8F).

To corroborate the association of UPF1 with the EBV and KSHV transactivator transcripts detected in our RIP-seq approach, we analyzed the UPF1-associated RNAs in several latently infected cells by qRT-PCR. We validated this assay by confirming the successful precipitation of phosphorylated (‘activated’) UPF1 by immunoblotting with anti-p-UPF1 (9A Fig) and the enrichment of the known cellular NMD-sensitive transcripts MAP3K14, GADD45B, and PDRG1 (41), but not the NMD-insensitive transcripts GAPDH, RPL32, or HPRT1, with UPF1 by qRT-PCR (9B-9G). In EBV⁺ AGS-EBV, Burkitt lymphoma AKBM and P3HR-1, as well as LCL cells we observed a striking enrichment of BRLF1 transcripts with UPF1 relative to the IgG control IP (FIG. 2D-2G). Other transcripts, such as cellular GAPDH or EBV BKRF1, BNRF1, BcLF1, and BLLF1 that were not enriched in our RIP-seq analysis, were not found to be significantly enriched by qRT-PCR either. We also observed robust and specific enrichment of Orf50 transcripts with UPF1 in KSHV⁺ HEK293T.rKSHV219 and BCBL1 cells, corroborating the RIP-seq results (FIGS. 2H and 2I).

Taken together, these results identify that the EBV BRLF1 and KSHV Orf50 transactivator-encoding transcripts are highly associated with UPF1 in several EBV- or KSHV-infected cell types.

### NMD Controls the Abundance of the Polycistronic EBV and KSHV Transactivator Transcripts.

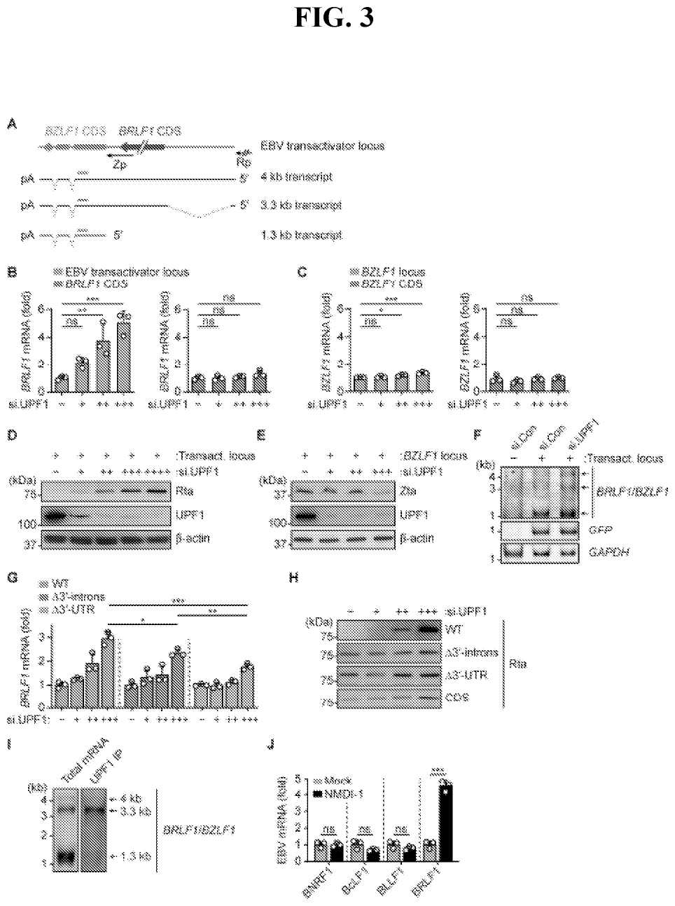

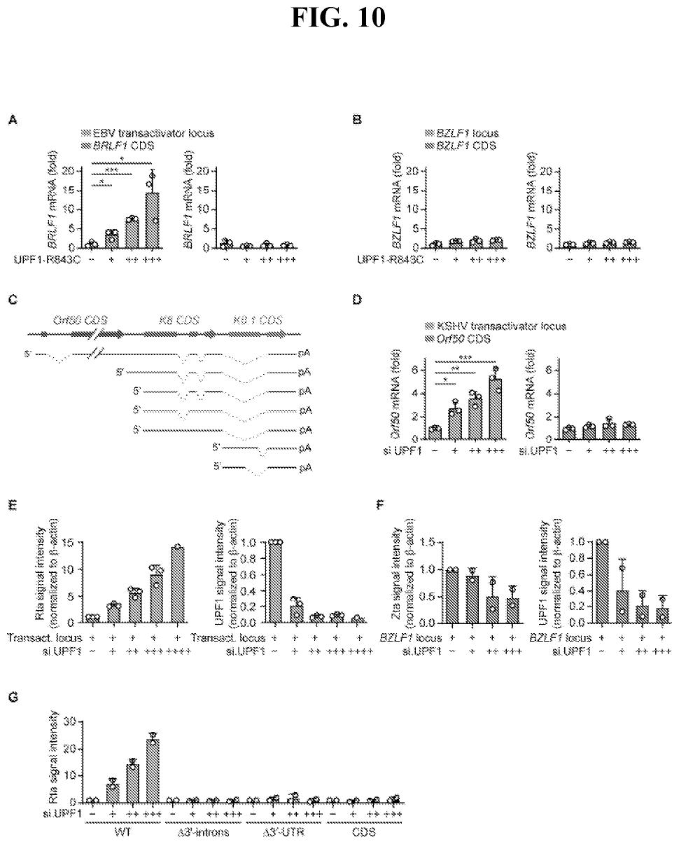

Expression of the viral Rta transactivator proteins encoded by the EBV BRLF1 and KSHV Orf50 transcripts is required and sufficient to induce the lytic reactivation cascade of these viruses (42, 43). Our observations that these transcripts associate with UPF1 led us to hypothesize that NMD-mediated degradation minimizes transactivator expression levels in latently infected cells to prevent gammaherpesvirus reactivation and, conversely, that NMD inhibition increases transactivator abundance resulting in viral reactivation. To test this hypothesis, we focused on the EBV transactivator locus, which is comprised of the BRLF1 and BZLF1 genes and gives rise to at least three different transcripts of 4 kb, 3.3 kb, and 1.3 kb in size that encode the EBV transactivator proteins Rta and/or Zta (FIG. 3A). To test the effect of NMD inhibition on EBV transactivator transcript and protein abundance in the absence of other viral proteins, the entire EBV transactivator locus consisting of BRLF1 and BZLF1 under control of the endogenous Rp promoter region (nucleotide −1 to −987 relative to the TSS for BRLF1 (44)) was expressed from a eukaryotic expression vector in naïve HEK293T cells. Cotransfected GFP-encoding plasmid served as an internal control to normalize for general differences in transfection and/or transcription efficiency between samples. We observed that NMD inhibition by either siRNA-mediated UPF1 silencing or overexpression of the dominant-negative UPF1-R843C mutant (45) led to a dose-dependent increase in BRLF1 transcript abundance (FIGS. 3B and 10A, left panels). In contrast, NMD inhibition did not significantly affect BRLF1 transcript abundance when expressed from a control plasmid that solely encoded the BRLF1 coding sequence (CDS) under control of the same viral Rp promoter (FIGS. 3B and 10A Fig, right panels). Expression of a construct encoding the BZLF1 locus under control of the endogenous Zp promoter (nucleotides −1 to −552), or a corresponding control plasmid encoding only the BZLF1 CDS, showed that monocistronic BZLF1 transcript levels were minimally affected by UPF1 silencing or UPF1-R843C overexpression (FIGS. 3C and 10B Fig). Analogously, we tested whether expression of the polycistronic KSHV Orf50 transcripts was suppressed by NMD by generating an expression plasmid encoding the entire KSHV transactivator locus consisting of Orf50, K8, and K8.1 under control of the endogenous Orf50 promoter (nucleotides −1 to −3079 relative to the Orf50 TSS (46)) (10C Fig). Similar to our results for EBV BRLF1, expression of this plasmid in UPF1-silenced cells resulted in a dose-dependent increase in Orf50 transcript abundance, whereas expression from an Orf50 CDS-only control plasmid was not affected (10D Fig).

Next, we tested whether the increase in EBV BRLF1 transcript levels upon NMD inhibition resulted in enhanced Rta protein abundance. Indeed, UPF1 depletion followed by transfection of the full-length transactivator locus expression plasmid caused an increase in EBV Rta abundance that correlated with the amount of transfected si.UPF1 (FIGS. 3D and 10E). Conversely, in line with our observation that the monocistronic BZLF1 transcript was not degraded by NMD, transfection of the plasmid encoding only the BZLF1 locus showed that Zta levels were not increased upon silencing of UPF1 (FIGS. 3E and 10F).

To corroborate these results, we depleted endogenous UPF1 from HEK293T cells using siRNA followed by transfection of the full length transactivator plasmid and then evaluated transcript abundance by Northern blotting using a probe that recognizes all three EBV transactivator transcripts (see FIG. 3A). In line with our earlier observations, the larger 3.3 and 4 kb polycistronic BRLF1 transcripts became more abundant following UPF1 silencing, whereas the abundance of the 1.3 kb BZLF1-encoding transcript was similar in control siRNA-transfected cells and UPF1-depleted cells (FIG. 3F). Taken together, these results show that NMD specifically induced the degradation of the viral polycistronic transactivator transcripts, which limits Rta gene and protein expression.

NMD Targets the Transactivator Transcripts by Recognizing Features in their 3′-UTR.

Our finding that the polycistronic BRLF1 transcripts, but not the BRLF1 CDS-only or the monocistronic BZLF1 transcripts, are sensitive to NMD suggests that the BRLF1 transcripts contain specific properties that facilitate recognition by the NMD machinery. The polycistronic BRLF1 transcripts display two primary NMD-inducing features: they possess a long 3′-UTR as well as two splice sites more than 55 nt downstream of the BRLF1 stop codon that can facilitate EJC deposition (see FIG. 3A). To determine which of these feature(s) sensitize(s) the BRLF1 transcripts to NMD-mediated degradation, we generated two mutant BRLF1 constructs lacking either the two 3′ introns that reside within the BZLF1 coding region (Δ3′-introns), or the entire 3′-UTR downstream of the BRLF1 stop codon (Δ3′-UTR). The transcripts derived from the plasmid lacking the two 3′ introns displayed reduced upregulation upon UPF1 depletion as compared to transcripts derived from the wildtype (WT) transactivator locus, while deletion of the entire 3′-UTR further reduced the effect of UPF1 silencing on transcript levels (FIG. 3G). In accord, whereas increased Rta protein abundance was observed upon UPF1 depletion when expressed from the full-length construct, Rta protein levels were only slightly affected by UPF1 depletion when expressed from plasmids lacking either the 3′ introns or the entire 3′-UTR, similarly to Rta expression from the CDS-only plasmid (FIGS. 3H and 10G Fig). These results suggest that primarily splicing-dependent EJC deposition in the 3′-UTR, but also the long 3′-UTR itself, are responsible for the sensitization of BRLF1 transcripts to NMD-mediated degradation.

### NMD Suppresses BRLF1 Expression in Virus-Infected Cells.

Next, we sought to corroborate these findings in virus-infected cells by determining which BRLF1 transcripts associate with UPF1 in AGS-EBV cells using Northern blotting. In line with our previous results, whereas all three major transcripts were identified in the total input mRNA pool, primarily the 3.3 kb and, to a much lesser extent, the 4 kb BRLF1-encoding transcripts were found to be associated with UPF1. In contrast, the 1.3 kb monocistronic BZLF1-encoding transcript was not enriched with UPF1 (FIG. 31).

We next asked whether the association of BRLF1 transcripts with UPF1 led to their degradation in virus-infected cells. Since BRLF1 transcript upregulation upon NMD inhibition would, through Rta transactivation activity, result in the induction of global lytic gene expression that would prevent us from evaluating a direct effect of NMD on transcript abundance, we treated AGS-EBV cells with TPA to induce equal BRLF1 and lytic gene expression in all samples; the abundance of viral lytic transcripts was then analyzed in the presence or absence of the small-molecule NMD inhibitor NMDI-1 (47). In these experimental settings, NMD inhibition by NMDI-1 treatment resulted in a significant increase in BRLF1 transcript abundance compared to the mock-treated control, whereas the levels for other EBV transcripts that were not enriched in our original RIP-seq screens, such as BNRF1, BcLF1, and BLLF1, were not significantly affected by NMDI-1 treatment (FIG. 3J). This strengthens the notion that BRLF1 transcripts are a direct target of the NMD machinery in EBV-infected cells, whereas other viral lytic transcripts such as BLLF1 and BcLF1 are not directly targeted by the NMD machinery and their upregulation in non-TPA treated cells (see FIGS. 1C and 1I) is very likely a consequence of the transactivation activity of upregulated Rta. Together, these results indicate that NMD targets and degrades BRLF1 transcripts during authentic EBV infection.

### Small-Molecule NMD Inhibitor NMDI-1 is a Potent Inducer of EBV and KSHV Reactivation.

EBV and KSHV are each responsible for a significant number of cancer cases each year. The currently available anti-herpesvirus drugs, such as ganciclovir, rely on expression of the viral kinases for their activation and therefore exclusively target reactivated cells (11). While lytic infection is increasingly appreciated to contribute to EBV and KSHV-associated malignancies, the far majority of virus-positive tumor cells are latently infected and thus insensitive to the currently available antiviral drugs (12). For this reason, there is a strong interest in the development of therapeutic strategies to induce reactivation and sensitize tumor cells to antiviral drugs. Since we identified the cellular NMD pathway as an important regulator of EBV and KSHV reactivation, we next sought to investigate the therapeutic potential of small-molecule NMD-inhibitory compounds by examining their ability to induce gammaherpesvirus reactivation. We focused on NMDI-1, a compound that inhibits NMD by blocking the interaction between SMG5 and UPF1 (47).

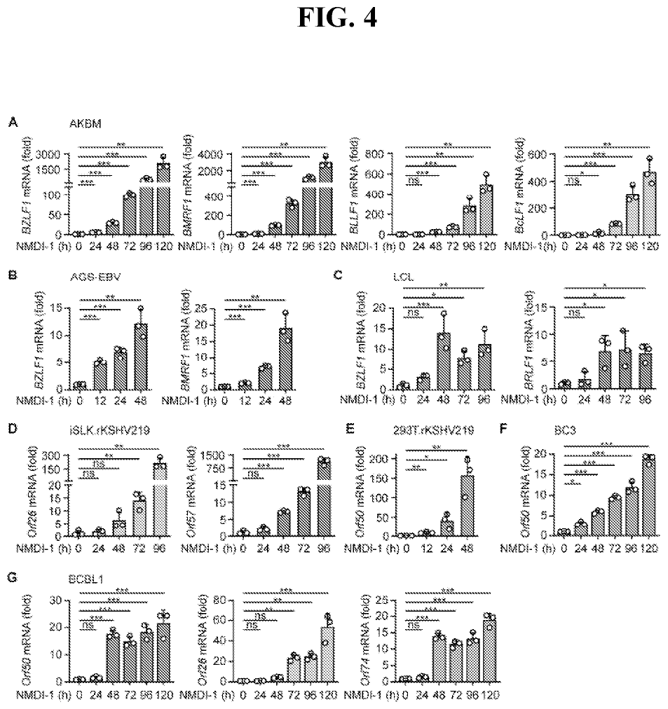

To test the effect of NMDI-1 on viral reactivation, we treated EBV⁺ AKBM cells with 25 μM NMDI-1 and assessed upregulation of the lytic genes BZLF1, BMRF1, BLLF1, and BcLF1 by qRT-PCR as a measure for viral reactivation (FIG. 4A). We observed that NMDI-1 treatment resulted in a striking induction of EBV lytic gene expression in AKBM cells. Similarly, a potent induction of lytic gene expression upon treatment with NMDI-1 was observed in AGS-EBV cells as well as in EBV-transformed LCL cells derived from healthy-donor primary B cells (FIGS. 4B and 4C). Of note, NMDI-1 at concentrations between 5 and 50 μM did not or only minimally affect cell viability (11A and 11B). Similarly to the effects seen on EBV reactivation, treatment of KSHV⁺ iSLK.rKSHV219, HEK293T.rKSHV219, and BCBL1 or BC3 PEL cells with NMDI-1 induced potent expression of the KSHV lytic transcripts Orf50, Orf26, Orf57 and/or Orf74 (FIG. 4D-4G). In conclusion, our results demonstrate that the small-molecule NMD-inhibitor NMDI-1 effectively induces EBV and KSHV reactivation in a variety of cell types.

NMD plays a well-documented role in regulating the abundance of a large variety of cellular transcripts; however, our knowledge of the interplay between NMD and viral infection, in particular infection with DNA viruses, remains rudimentary. Along these lines, although recent reports have revealed a contribution of NMD to RNA virus infections, only few bona-fide viral NMD targets have been identified (21-27). In this study, we show that NMD targets the spliced, polycistronic EBV and KSHV transactivator-encoding transcripts for degradation through the recognition of NMD-inducing features in their 3′-UTRs; this ultimately keeps the abundance of the EBV and KSHV Rta proteins to a minimum, thereby suppressing virus reactivation. Our findings thus identify NMD as a key regulator of oncogenic DNA virus infection.

The biphasic life cycle consisting of latency establishment in long-lived cells and the occasional reactivation that results in production of viral progeny is a hallmark of herpesvirus infection. Given the central role of the Rta transactivator proteins of EBV and KSHV in initiating lytic reactivation, it is not surprising that their expression is extensively regulated. The cotranslational regulation of Rta mRNA stability by NMD that we identified here provides an extra layer of control in addition to the epigenetic, transcriptional, and post-transcriptional regulatory mechanisms that have been reported previously (37, 48-50). While the present invention is not limited to any particular mechanism, and an understanding of the mechanism is not necessary to practice the invention, these results suggest that NMD prevents viral reactivation by degrading transactivator transcripts that are produced at low levels in latently infected cells. The strong lytic reactivation that we observed upon inhibition of NMD, to a similar extent as treatment with potent chemical inducers such as sodium butyrate, underscores the importance of NMD in preserving EBV and KSHV latency.

While the majority of cells in EBV- and KSHV-associated cancers are latently infected, only few other, non-tumor cells are typically virus-positive. Therefore, therapeutic induction of lytic reactivation can be used to specifically sensitize tumor cells to antiviral drugs and to activate cytotoxic T-lymphocyte responses that are typically directed against lytic antigens (12, 48, 58, 59). In this example, we observed a strong induction of EBV and KSHV reactivation by the small-molecule NMD-inhibitor NMDI-1 in a variety of cell types, even those that are notoriously refractory to reactivation. Concentrations of NMDI-1 below those used in our study have been successfully used in in vivo studies without apparent toxicity (60, 61). Moreover, it was recently reported that modest NMD inhibition does not have an appreciable negative impact on overall health in mice (62). Together, this suggests that NMD inhibition therapeutically induces viral reactivation in the treatment of EBV and KSHV-associated malignancies.

### REFERENCES

1. Mesri E A, Cesarman E, Boshoff C. Kaposi's sarcoma and its associated herpesvirus. Nature Reviews Cancer. 2010; 10:707.
2. Cesarman E. Gammaherpesviruses and Lymphoproliferative Disorders. Annual Review of Pathology: Mechanisms of Disease. 2014; 9(1):349-72.
3. Parkin D M. The global health burden of infection-associated cancers in the year 2002. International Journal of Cancer. 2006; 118(12):3030-44.
4. Cavallin L E, Goldschmidt-Clermont P, Mesri E A. Molecular and Cellular Mechanisms of KSHV Oncogenesis of Kaposi's Sarcoma Associated with HIV/AIDS. PLOS Pathogens. 2014; 10(7):e1004154.
5. Bouvard V, Baan R, Straif K, Grosse Y, Secretan B, Ghissassi F E, et al. A review of human carcinogens—Part B: biological agents. The Lancet Oncology. 2009; 10(4):321-2.
6. Lieberman P M. Keeping it quiet: chromatin control of gammaherpesvirus latency. Nature Reviews Microbiology. 2013; 11(12):863-75.
7. Yuan J, Cahir-McFarland E, Zhao B, Kieff E. Virus and Cell RNAs Expressed during Epstein-Barr Virus Replication. Journal of Virology. 2006; 80(5):2548-65.
8. He M, Cheng F, da Silva S R, Tan B, Sorel O, Gruffaz M, et al. Molecular Biology of KSHV in Relation to HIV/AIDS-Associated Oncogenesis. Cancer Treat Res. 2019; 177:23-62.
9. Ersing I, Nobre L, Wang L W, Soday L, Ma Y, Paulo J A, et al. A Temporal Proteomic Map of Epstein-Barr Virus Lytic Replication in B Cells. Cell Reports. 2017; 19(7):1479-93.
10. Israel B F, Kenney S C. Virally targeted therapies for EBV-associated malignancies. Oncogene. 2003; 22(33):5122-30.
11. Meng Q, Hagemeier S R, Fingeroth J D, Gershburg E, Pagano J S, Kenney S C. The Epstein-Barr virus (EBV)-encoded protein kinase, EBV-PK, but not the thymidine kinase (EBV-TK), is required for ganciclovir and acyclovir inhibition of lytic viral production. Journal of Virology. 2010; 84(9):4534-42.
12. Manners O, Murphy J C, Coleman A, Hughes D J, Whitehouse A. Contribution of the KSHV and EBV lytic cycles to tumourigenesis. Current Opinion in Virology. 2018; 32:60-70.
13. Münz C. Latency and lytic replication in Epstein-Barr virus-associated oncogenesis. Nature Reviews Microbiology. 2019; 17(11):691-700.
14. Kurosaki T, Popp M W, Maquat L E. Quality and quantity control of gene expression by nonsense-mediated mRNA decay. Nature Reviews Molecular Cell Biology. 2019; 20(7):406-20.
15. Lykke-Andersen S, Jensen T H. Nonsense-mediated mRNA decay: an intricate machinery that shapes transcriptomes. Nature Reviews Molecular Cell Biology. 2015; 16:665.
16. Karousis E D, Nasif S, Milhlemann O. Nonsense-mediated mRNA decay: novel mechanistic insights and biological impact. Wiley Interdisciplinary Reviews: RNA. 2016; 7(5):661-82.
17. Hurt J A, Robertson A D, Burge C B. Global analyses of UPF1 binding and function reveal expanded scope of nonsense-mediated mRNA decay. Genome Research. 2013; 23(10):1636-50.
18. Le Hir H, Gatfield D, Izaurralde E, Moore M J. The exon-exon junction complex provides a binding platform for factors involved in mRNA export and nonsense-mediated mRNA decay. The EMBO Journal. 2001; 20(17):4987-97.
19. Lykke-Andersen J, Shu M D, Steitz J A. Communication of the Position of Exon-Exon Junctions to the mRNA Surveillance Machinery by the Protein RNPS1. Science. 2001; 293(5536):1836-9.
20. Yepiskoposyan H, Aeschimann F, Nilsson D, Okoniewski M, Malemann O. Autoregulation of the nonsense-mediated mRNA decay pathway in human cells. RNA. 2011; 17:2108-18.
21. Balistreri G, Horvath P, Schweingruber C, Zund D, McInerney G, Merits A, et al. The Host Nonsense-Mediated mRNA Decay Pathway Restricts Mammalian RNA Virus Replication. Cell Host & Microbe. 2014; 16(3):403-11.
22. Wada M, Lokugamage K G, Nakagawa K, Narayanan K, Makino S. Interplay between coronavirus, a cytoplasmic RNA virus, and nonsense-mediated mRNA decay pathway. Proceedings of the National Academy of Sciences. 2018; 115(43):E10157-E66.
23. Li M, Johnson J R, Truong B, Kim G, Weinbren N, Dittmar M, et al. Identification of antiviral roles for the exon-junction complex and nonsense-mediated decay in flaviviral infection. Nature Microbiology. 2019; 4(6):985-95.
24. Rao S, Amorim R, Niu M, Temzi A, Mouland A J. The RNA surveillance proteins UPF1, UPF2 and SMG6 affect HIV-1 reactivation at a post-transcriptional level. Retrovirology. 2018; 15(1):42.
25. Ramage Holly R, Kumar G R, Verschueren E, Johnson Jeffrey R, Von Dollen J, Johnson T, et al. A Combined Proteomics/Genomics Approach Links Hepatitis C Virus Infection with Nonsense-Mediated mRNA Decay. Molecular Cell. 2015; 57(2):329-40.
26. Garcia D, Garcia S, Voinnet O. Nonsense-Mediated Decay Serves as a General Viral Restriction Mechanism in Plants. Cell Host & Microbe. 2014; 16(3):391-402.
27. Fontaine K A, Leon K E, Khalid M M, Tomar S, Jimenez-Morales D, Dunlap M, et al. The Cellular NMD Pathway Restricts Zika Virus Infection and Is Targeted by the Viral Capsid Protein. mBio. 2018; 9(6):e02126-18.
28. Arias C, Weisburd B, Stern-Ginossar N, Mercier A, Madrid A S, Bellare P, et al. KSHV 2.0: A Comprehensive Annotation of the Kaposi's Sarcoma-Associated Herpesvirus Genome Using Next-Generation Sequencing Reveals Novel Genomic and Functional Features. PLOS Pathogens. 2014; 10(1):e1003847.
29. O'Grady T, Cao S, Strong M J, Concha M, Wang X, BonDurant S S, et al. Global bidirectional transcription of the Epstein-Barr virus genome during reactivation. Journal of Virology. 2014; 88(3):1604-16.
30. Zheng Z M. Split genes and their expression in Kaposi's sarcoma-associated herpesvirus. Reviews in Medical Virology. 2003; 13(3):173-84.
31. Zhu Y, Huang Y, Jung J U, Lu C, Gao S J. Viral miRNA targeting of bicistronic and polycistronic transcripts. Current Opinion in Virology. 2014; 7:66-72.
32. Marquitz A R, Mathur A, Shair K H Y, Raab-Traub N. Infection of Epstein-Barr virus in a gastric carcinoma cell line induces anchorage independence and global changes in gene expression. Proceedings of the National Academy of Sciences. 2012; 109(24):9593-8.
33. Park E, Maquat L E. Staufen-mediated mRNA decay. Wiley Interdisciplinary Reviews: RNA. 2013; 4(4):423-35.
34. Vieira J, O'Hearn P M. Use of the red fluorescent protein as a marker of Kaposi's sarcoma-associated herpesvirus lytic gene expression. Virology. 2004; 325(2):225-40.
35. Ressing M E, Keating S E, van Leeuwen D, Koppers-Lalic D, Pappworth I Y, Wiertz E J H J, et al. Impaired Transporter Associated with Antigen Processing-Dependent Peptide Transport during Productive EBV Infection. The Journal of Immunology. 2005; 174(11):6829-38.
36. Takada K, Ono Y. Synchronous and sequential activation of latently infected Epstein-Barr virus genomes. Journal of Virology. 1989; 63(1):445-9.
37. Anej a K K, Yuan Y. Reactivation and Lytic Replication of Kaposi's Sarcoma-Associated Herpesvirus: An Update. Frontiers in Microbiology. 2017; 8:613.
38. Kalla M, Hammerschmidt W. Human B cells on their route to latent infection—Early but transient expression of lytic genes of Epstein-Barr virus. European Journal of Cell Biology. 2012; 91(1):65-9.
39. Colombo M, Karousis E D, Bourquin J, Bruggmann R, Malemann O. Transcriptome-wide identification of NMD-targeted human mRNAs reveals extensive redundancy between SMG6- and SMG7-mediated degradation pathways. RNA. 2017; 23(2):189-201.
40. Kurosaki T, Li W, Hoque M, Popp MWL, Ermolenko D N, Tian B, et al. A post-translational regulatory switch on UPF1 controls targeted mRNA degradation. Genes & Development. 2014; 28(17):1900-16.
41. Mendell J T, Sharifi N A, Meyers J L, Martinez-Murillo F, Dietz H C. Nonsense surveillance regulates expression of diverse classes of mammalian transcripts and mutes genomic noise. Nature Genetics. 2004; 36:1073.
42. Feederle R, Kost M, Baumann M, Janz A, Drouet E, Hammerschmidt W, et al. The Epstein-Barr virus lytic program is controlled by the co-operative functions of two transactivators. The EMBO Journal. 2000; 19(12):3080-9.
43. Staudt M R, Dittmer D P. The Rta/Orf50 transactivator proteins of the gamma-herpesviridae. Curr Top Microbiol Immunol. 2007; 312:71-100.
44. Bhende P M, Seaman W T, Delecluse H J, Kenney S C. BZLF1 Activation of the Methylated Form of the BRLF1 Immediate-Early Promoter Is Regulated by BZLF1 Residue 186. Journal of Virology. 2005; 79(12):7338-48.
45. Sun X, Perlick H A, Dietz H C, Maquat L E. A mutated human homologue to yeast Upf1 protein has a dominant-negative effect on the decay of nonsense-containing mRNAs in mammalian cells. Proceedings of the National Academy of Sciences. 1998; 95(17):10009-14.
46. Wilson S J, Tsao E H, Webb BLJ, Ye H, Dalton-Griffin L, Tsantoulas C, et al. X Box Binding Protein XBP-1s Transactivates the Kaposi's Sarcoma-Associated Herpesvirus (KSHV) ORF50 Promoter, Linking Plasma Cell Differentiation to KSHV Reactivation from Latency. Journal of Virology. 2007; 81(24):13578-86.
47. Durand S, Cougot N, Mahuteau-Betzer F, Nguyen C H, Grierson D S, Bertrand E, et al. Inhibition of nonsense-mediated mRNA decay (NMD) by a new chemical molecule reveals the dynamic of NMD factors in P-bodies. The Journal of Cell Biology. 2007; 178(7):1145-60.
48. Kenney S C, Mertz J E. Regulation of the latent-lytic switch in Epstein-Barr virus. Seminars in Cancer Biology. 2014; 26:60-8.
49. Chen H S, Lu F, Lieberman P M. Epigenetic regulation of EBV and KSHV latency. Current Opinion in Virology. 2013; 3(3):251-9.
50. Frost T C, Gewurz B E. Epigenetic crossroads of the Epstein-Barr virus B-cell relationship. Current Opinion in Virology. 2018; 32:15-23.
51. Zhao Y, Ye X, Shehata M, Dunker W, Xie Z, Karijolich J. The RNA quality control pathway nonsense-mediated mRNA decay targets cellular and viral RNAs to restrict KSHV. Nature Communications. 2020; 11(1):3345.
52. Guo R, Jiang C, Zhang Y, Govande A, Trudeau S J, Chen F, et al. MYC Controls the Epstein-Barr Virus Lytic Switch. Molecular Cell. 2020; 78(4):653-69.e8.
53. Fiorini F, Robin J P, Kanaan J, Borowiak M, Croquette V, Le Hir H, et al. HTLV-1 Tax plugs and freezes UPF1 helicase leading to nonsense-mediated mRNA decay inhibition. Nature Communications. 2018; 9(1):431.
54. Nakano K, Ando T, Yamagishi M, Yokoyama K, Ishida T, Ohsugi T, et al. Viral interference with host mRNA surveillance, the nonsense-mediated mRNA decay (NMD) pathway, through a new function of HTLV-1 Rex: implications for retroviral replication. Microbes and Infection. 2013; 15(6-7):491-505.
55. Toma K G, Rebbapragada I, Durand S, Lykke-Andersen J. Identification of elements in human long 3′ UTRs that inhibit nonsense-mediated decay. RNA. 2015; 21(5):887-97.
56. Pereverzev A P, Gurskaya N G, Ermakova G V, Kudryavtseva E I, Markina N M, Kotlobay A A, et al. Method for quantitative analysis of nonsense-mediated mRNA decay at the single cell level. Scientific Reports. 2015; 5:7729.
57. Nguyen L S, Wilkinson M F, Gecz J. Nonsense-mediated mRNA decay: Inter-individual variability and human disease. Neuroscience & Biobehavioral Reviews. 2014; 46:175-86.
58. Novalie Z, Verkuijlen S A W M, Verlaan M, Eersels J L H, de Greeuw I, Molthoff CFM, et al. Cytolytic virus activation therapy and treatment monitoring for Epstein-Barr virus associated nasopharyngeal carcinoma in a mouse tumor model. Journal of Medical Virology. 2017; 89(12):2207-16.
59. Zhou F, Shimoda M, Olney L, Lyu Y, Tran K, Jiang G, et al. Oncolytic Reactivation of KSHV as a Therapeutic Approach for Primary Effusion Lymphoma. Molecular Cancer Therapeutics. 2017; 16(11):2627-38.
60. Keeling K M, Wang D, Dai Y, Murugesan S, Chenna B, Clark J, et al. Attenuation of Nonsense-Mediated mRNA Decay Enhances In Vivo Nonsense Suppression. PLOS ONE. 2013; 8(4):e60478.
61. Popp M W, Maquat L E. Attenuation of nonsense-mediated mRNA decay facilitates the response to chemotherapeutics. Nature Communications. 2015; 6:6632.
62. Echols J, Siddiqui A, Dai Y, Havasi V, Sun R, Kaczmarczyk A, et al. A regulated NMD mouse model supports NMD inhibition as a viable therapeutic option to treat genetic diseases. Disease Models & Mechanisms. 2020:dmm.044891.
63. van de Weijer M L, Bassik M C, Luteijn R D, Voorburg C M, Lohuis MAM, Kremmer E, et al. A high-coverage shRNA screen identifies TMEM129 as an E3 ligase involved in ER-associated protein degradation. Nature Communications. 2014; 5:3832.
64. Afgan E, Baker D, Batut B, van den Beek M, Bouvier D, tech M, et al. The Galaxy platform for accessible, reproducible and collaborative biomedical analyses: 2018 update. Nucleic Acids Research. 2018; 46(W1):W537-W44.
65. Kim D, Langmead B, Salzberg S L. HISAT: a fast spliced aligner with low memory requirements. Nature Methods. 2015; 12(4):357-60.
66. Liao Y, Smyth G K, Shi W. featureCounts: an efficient general purpose program for assigning sequence reads to genomic features. Bioinformatics. 2013; 30(7):923-30.
67. Love M I, Huber W, Anders S. Moderated estimation of fold change and dispersion for RNA-seq data with DESeq2. Genome Biology. 2014; 15(12):550.
68. Li H. A statistical framework for SNP calling, mutation discovery, association mapping and population genetical parameter estimation from sequencing data. Bioinformatics. 2011; 27(21):2987-93.

All publications and patents mentioned in the specification and/or listed below are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope described herein.

We claim:

1. A method comprising:
a) administering an NMD inhibitor to a subject infected with a human herpesvirus, and
b) administering a herpesvirus antiviral agent to said subject: prior to, after, or with said administering of said NMD inhibitor.

2. The method of claim 1, wherein said subject further has cancer caused by said human herpesvirus, and wherein the method further comprises: c) administering a cancer treatment agent to said subject: prior to, after, or with said administering of said NMD inhibitor.

3. The method of claim 1, wherein said cancer treatment agent comprises an immunomodulatory agent.

4. The method of claim 1, wherein said human herpesvirus is EBV (Epstein-Barr virus).

5. The method of claim 1, wherein said human herpesvirus is KSHV (Kaposi's sarcoma-associated herpesvirus).

6. The method of claim 1, wherein said human herpesvirus is selected from the group consisting of: HSV-1 (herpes simplex virus 1), HSV-2 (herpes simplex virus 2), VZV (varicella zoster virus), CMV (cytomegalovirus), HHV6A (human herpesvirus 6A), HHV6B (human herpesvirus 6B), and HHV7 (human herpesvirus 7).

7. The method of claim 1, wherein said NMD inhibitor is selected from the group consisting of: NMDI-1, NMDI-14, and VGI.

8. The method of claim 1, wherein said herpesvirus antiviral comprises ganciclovir.

9. The method of claim 1, wherein said subject is a human.

10. A system or kit comprising:
a) an NMD inhibitor, and
b) a herpesvirus antiviral.

11. The system or kit of claim 10, wherein said NMD inhibitor is selected from the group consisting of: NMDI-1, NMDI-14, and VGI.

12. The system or kit of claim 10, wherein said herpesvirus antiviral comprises ganciclovir or valganciclovir.

13. The system or kit of claim 10, further comprising: a cancer treatment agent.

14. The system or kit of claim 13, wherein said cancer treatment agent comprises an immunomodulatory agent.

15. A composition comprising:
a) an NMD inhibitor, and
b) a herpesvirus antiviral.

16. The composition of claim 15, wherein said NMD inhibitor is selected from the group consisting of: NMDI-1, NMDI-14, and VGI.

17. The composition of claim 15, wherein said herpesvirus antiviral comprises ganciclovir or valganciclovir.

18. The composition of claim 15, further comprising: a cancer treatment agent.

19. The composition of claim 138, wherein said cancer treatment agent comprises an immunomodulatory agent.
